(19) United States
(12) Patent Application Publication
(10) Pub. No.: US 2020/0094236 A1
(43) Pub. Date: Mar. 26, 2020

(54) FILTER

(71) Applicant: FUJI XEROX CO., LTD., Tokyo (JP)

(72) Inventors: Takeshi IWANAGA, Kanagawa (JP); Hiroyoshi OKUNO, Kanagawa (JP); Hideaki YOSHIKAWA, Kanagawa (JP); Sakae TAKEUCHI, Kanagawa (JP); Yuka ZENITANI, Kanagawa (JP)

(73) Assignee: FUJI XEROX CO., LTD., Tokyo (JP)

(21) Appl. No.: 16/384,018

(22) Filed: Apr. 15, 2019

(30) Foreign Application Priority Data
Sep. 26, 2018 (JP) 2018-179874

(51) Int. Cl. B01J 35/00; B01D 39/18; B01J 35/02; B01J 35/04; B01J 21/06

(52) U.S. Cl. CPC B01J 35/004; B01D 39/18; A61L 9/16; B01J 35/04; B01J 21/063; B01J 35/023

(57) ABSTRACT

A filter includes a filter body containing a paper sheet having a surface roughness of 1.0 μm or more and 10.0 μm or less, and a photocatalyst particle held on the filter body and having a surface to which a metal compound having a metal atom and a hydrocarbon group is bonded via an oxygen atom. The photocatalyst particle is formed of a titanium compound particle having absorption at wavelengths of 450 nm and 750 nm in a visible absorption spectrum.

### CROSS-REFERENCE TO RELATED APPLICATIONS

This application is based on and claims priority under 35 USC 119 from Japanese Patent Application No. 2018-179874 filed Sep. 26, 2018.

### BACKGROUND

### (i) Technical Field

The present disclosure relates to a filter.

### (ii) Related Art

One of known air or water purification methods involves decomposition and removal of contaminants and odor substances by means of the photocatalytic effect. There have been proposed various purification apparatuses that include, for example, a filter having photocatalyst particles, such as those formed of anatase titanium oxide, on the surface of a fiber, such as a nonwoven fabric. The irradiation of the filter with UV rays decomposes contaminants and odor substances adsorbed onto the filter. Various filters are proposed as filters having photocatalyst particles.

For example, Japanese Laid Opened Patent Application Publication No. 2011-218764 discloses a “plant fiber-based thermal insulation board including a plant fiber-based core material and a plant fiber element, wherein the core material includes a thin wall and pores that are defined by the thin wall and penetrate in the thickness direction of the plant fiber-based thermal insulation board, the core material is disposed in the entire region of the plant fiber-based thermal insulation board, and the plant fiber element is made of a plant fiber that is attached to the periphery of the core material and the inside of the pores and subjected to compression”.

Japanese Laid Opened Patent Application Publication No. 2006-305547 discloses a “porous composite material including a porous body (2) having plural pores (2a) extending from the front surface to the back surface and a titanium dioxide-containing film (3) deposited on 95% to 100% of the total surface of the porous body (2) including the inner surfaces of the pores (2a)”.

Japanese Laid Opened Patent Application Publication No. 2005-199266 discloses a “honeycomb air filter including a honeycomb paper filter, wherein a chemical substance is attached to the honeycomb paper filter, and the honeycomb paper filter is used for filtration”.

Japanese Laid Opened Patent Application Publication No. 2004-330088 discloses an “air cleaner using photocatalyst Japanese paper and including a light-transmissible case having an air passage inside, a helical filter that is formed of photocatalyst Japanese paper and axially and rotatably supported in the air passage of the light-transmissible case, an air blowing unit at one end of the case, and an air exhausting unit at the other end of the case”.

Japanese Laid Opened Patent Application Publication No. 2003-236391 discloses a “photocatalyst paper material formed by mixing a base fiber and a photocatalyst-carrying inorganic fiber, wherein the contact between the photocatalyst and the base fiber is minimized to prevent the deterioration of the base and provide the effect of decomposing and removing hazardous substances and the effect of improving the living environment due to the photocatalyst”.

Japanese Laid Opened Patent Application Publication No. 2002-204929 discloses a “gas-phase environmental cleaning module in which a photocatalyst containing titania as a main component is fibrous and immobilized in a space at a low density without using a binder”.

Japanese Laid Opened Patent Application Publication No. 2002-194667 discloses a “woven or knitted fabric formed of a paper yarn obtained by covering a fibrous yarn with a fibrous paper, wherein a negative ion-generating substance is present in at least one of the fibrous yarn and the fibrous paper at a proportion of 0.1% to 10% by weight relative to the weight of the woven or knitted fabric”.

Japanese Laid Opened Patent Application Publication No. 2002-194642 discloses a “paper yarn woven or knitted fabric including a paper yarn having a core formed of a fibrous yarn and covered with a sheath of fibrous paper or a paper yarn formed by blending and twisting a fibrous yarn and a fibrous paper”.

Japanese Laid Opened Patent Application Publication No. 2002-161496 discloses an “immobilized photocatalyst-containing paper that contains photocatalyst-immobilized diatomaceous earth”.

Japanese Laid Opened Patent Application Publication No. 2002-61098 discloses a “photocatalyst pulp composition having a water content of 10% by weight or more and containing a pulp and/or paper fiber to which inorganic fine particles having a photocatalytic activity are affixed and/or immobilized”.

Japanese Laid Opened Patent Application Publication No. 2000-199174 discloses a “metal fiber having an inorganic substance layer having a photocatalytic function”.

Japanese Laid Opened Patent Application Publication No. 11-57360 discloses a “dust filter including plural filter elements formed by cutting a honeycomb core multilayer in parallel at a predetermined angle of inclination so as to have a predetermined thickness, wherein the filter elements are sequentially stacked in the thickness direction with the angle of inclination alternately reversed in such a manner that a passage formed by cells of the filter elements in the layers is bent in a <-shape or a zigzag shape”.

Japanese Laid Opened Patent Application Publication No. 2006-276137 discloses a “display panel formed by attaching a printed print sheet to a display panel base plate obtained by attaching a paperboard to at least one side of a paper core, wherein the display panel base plate includes a white paperboard as the paperboard, and the print sheet has an adhesive layer on one side of the substrate”.

Japanese Laid Opened Patent Application Publication No. 2009-263820 discloses a “stain-proof antibacterial agent for fiber products obtained by mixing a polyacrylic acid having a molecular weight of 20,000 or less and/or an alkali metal salt thereof, a titanate coupling agent, and water”.

### SUMMARY

A known filter having photocatalyst particles may undergo deterioration of deodorizing performance over time as a result of the detachment of the photocatalyst particles from the filter and the degradation of the material of the filter due to the photocatalytic function of the photocatalyst particles.

Aspects of non-limiting embodiments of the present disclosure relate to a filter having higher deodorization maintainability than a filter including a paper filter body having a surface roughness of less than 1.0 μm and more than 10.0 μm, and a photocatalyst particle held on the paper filter body and having a surface to which a metal compound having a metal atom and a hydrocarbon group is bonded via an oxygen atom, the photocatalyst particle having absorption at wavelengths of 450 nm and 750 nm in a visible absorption spectrum.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided a filter including a filter body containing a paper sheet having a surface roughness of 1.0 μm or more and 10.0 μm or less, and each photocatalyst particle of photocatalyst particles, held on the filter body and having a surface to which a metal compound having a metal atom and a hydrocarbon group is bonded via an oxygen atom, the photocatalyst particle being formed of a titanium compound particle having absorption at wavelengths of 450 nm and 750 nm in a visible absorption spectrum.

### BRIEF DESCRIPTION OF THE DRAWINGS

Exemplary embodiments of the present disclosure will be described in detail based on the following figures, wherein:

FIG. 1 is a schematic perspective view of one example of a filter according to an exemplary embodiment;

FIG. 2 is a schematic perspective view of another example of the filter according to the exemplary embodiment;

FIG. 3 is a schematic enlarged view of another example of the filter according to the exemplary embodiment;

FIG. 4 is a schematic structural view for illustrating the state of photocatalyst particles held on the filter;

FIG. 5 is a schematic structural view for illustrating the state of the photocatalyst particles held on the filter; and

FIG. 6 illustrates examples of the elemental profiles of silica-titania composite particles and includes, in sequence from top, an elemental profile for titanium, an elemental profile for silicon, and an elemental profile for carbon.

### DETAILED DESCRIPTION

Exemplary embodiments of the present disclosure will be described below. The following description and Examples are provided to illustrate exemplary embodiments of the present disclosure but are not intended to limit the scope of the present disclosure.

In this specification, the amount of a component in a composition refers to, when there are several substances corresponding to the component in the composition, the total amount of the substances present in the composition, unless otherwise specified.

The term “step” not only includes an independent step but also includes a step that, even when cannot be clearly distinguished from other steps, accomplishes an intended purpose of the step.

The term “XPS” is an acronym for X-ray Photoelectron Spectroscopy.

A filter according to an exemplary embodiment includes a paper filter body having a surface roughness of 1.0 μm or more and 10.0 μm or less, and a photocatalyst particle held on the paper filter body.

The photocatalyst particle is formed of a titanium compound particle (hereinafter may also be referred to as a “specific titanium compound particle”) having a surface to which a metal compound having a metal atom and a hydrocarbon group is bonded via an oxygen atom, wherein the titanium compound particle has absorption at wavelengths of 450 nm and 750 nm in the visible absorption spectrum.

The filter according to the exemplary embodiment has high deodorization maintainability. The reason for this is assumed as described below.

A known filter having photocatalyst particles may undergo deterioration of deodorizing performance over time as a result of the detachment of the photocatalyst particles from the filter and the degradation of the material of the filter due to the photocatalytic function of the photocatalyst particles.

Specifically, when the filter is made of paper, regions of the filter in contact with the photocatalyst particles are gradually degraded because of the characteristics of the material and the photocatalytic function of the photocatalyst particles. This degradation may cause detachment of the photocatalyst particles and defects in the filter itself and may result in deterioration of deodorizing performance over time. The deodorizing performance may deteriorate due to the use of the filter because the photocatalyst particles have a weak adhesion strength.

However, the “specific titanium compound particle” serving as a photocatalyst particle has a surface to which a metal compound having a metal atom and a hydrocarbon group is bonded via an oxygen atom. The specific titanium compound particle thus has the surface with high adhesiveness, and the specific titanium compound particle strongly adhere to (are firmly held on) the paper filter body. In addition, the specific titanium compound particle has hydrophobicity and thus has high dispersibility.

Furthermore, the specific titanium compound particle has absorption at wavelengths of 450 nm and 750 nm in the visible absorption spectrum and has a high photocatalytic function in the visible light region.

However, even when the photocatalyst particle having high dispersibility and adhesiveness and having a high photocatalytic function is held on the paper filter body, the following phenomena may occur depending on the surface profile of the paper filter body and depending on the surface profile of the filter: 1) it is difficult to increase the amount of the held photocatalyst particle, 2) the photocatalyst particle are embedded even when the amount of the held photocatalyst particle is excessively increased, and 3) the photocatalyst particle is detached. Therefore, the filter may have low deodorization maintainability.

However, when the photocatalyst particle formed of the specific titanium compound particle is held on a paper filter body having a surface roughness of 1.0 μm or more and 10 μm or less, the following phenomena are prevented or reduced: 1) an increase in the amount of the held photocatalyst particle, embedding of the photocatalyst particles, and detachment of the photocatalyst particle.

This may be because the size of the photocatalyst particle are in the optimum range for deposition in recesses so that the photocatalyst particle is held on the paper filter body, and embedding and detachment of the photocatalyst particle can be reduced or prevented without increasing the amount of the held photocatalyst particle. As a result, the photocatalyst particle can be firmly held on the paper filter body.

Since the photocatalyst particle formed of the specific titanium compound particle has a “metal compound having a metal atom and a hydrocarbon group” on its surface, the photocatalyst effect does not directly act on the material of the paper filter body, and the degradation of the material is unlikely to occur.

As a result, the filter may have high deodorization maintainability.

On the basis of the foregoing description, the filter according to the exemplary embodiment is assumed to have high deodorization maintainability.

Since the photocatalyst particle formed of the specific titanium compound particle in the filter according to the exemplary embodiment has the surface with high adhesiveness, a deodorant component is strongly adsorbed to the photocatalyst particle. Therefore, the filter according to the exemplary embodiment has high deodorizing performance.

An example of the filter according to the exemplary embodiment will be described below with reference to the drawings.

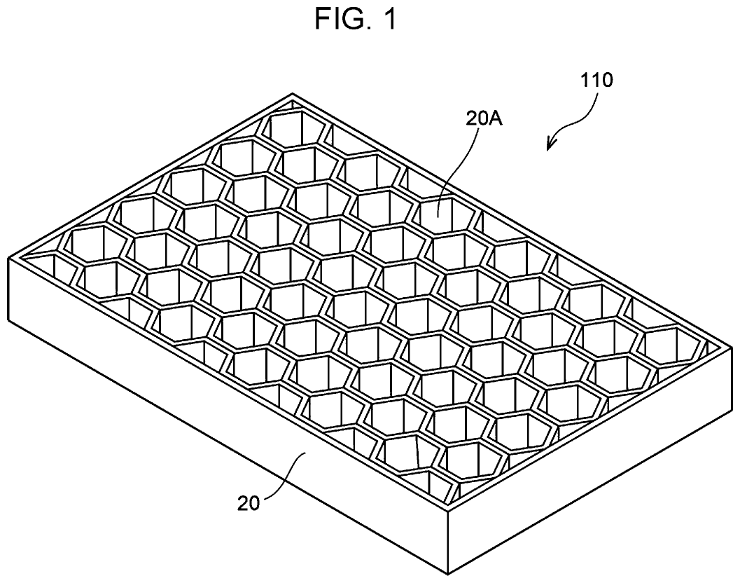

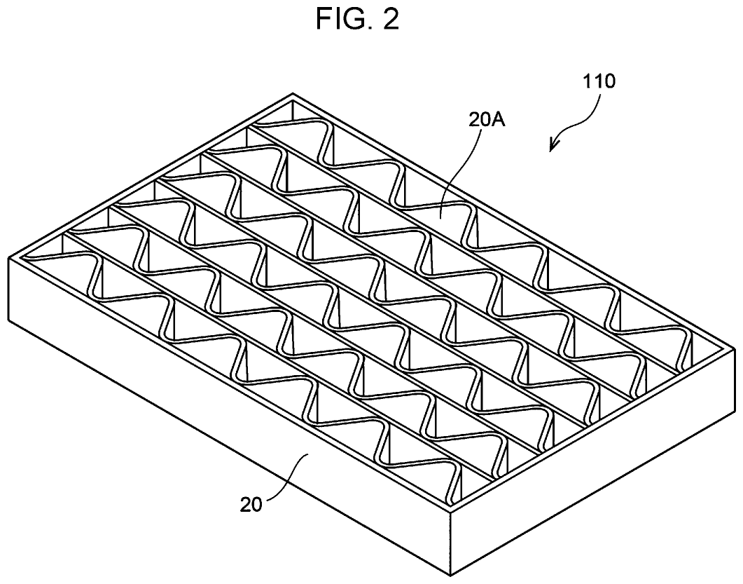

As illustrated in FIG. 1 and FIG. 2, a filter 101 according to an exemplary embodiment includes a paper filter body 20, and photocatalyst particles 10 held on the paper filter body 20.

### Paper Filter Body 20

A paper filter body 20 (hereinafter may also be referred to as a “filter body 20”) is a filter that will have the photocatalyst particles 10.

The filter body 20 is an air-permeable, liquid-permeable member made of paper and having a filter function.

The surface roughness of the filter body 20 is 1.0 μm or more and 10.0 μm or less. To improve deodorization maintainability, the surface roughness of the filter body 20 is preferably 1.5 μm or more and 8.0 μm or less and more preferably 2.0 μm or more and 5.0 μm or less.

When the filter body 20 is a monolayer or multilayer paper sheet, the surface roughness of the filter body 20 refers to the surface roughness of the monolayer or multilayer paper sheet. When the filter body 20 is a member having an opening, the surface roughness of the filter body 20 refers to the surface roughness of the wall surface of the opening.

The surface roughness of the filter body 20 is determined in accordance with the method described in JIS P 8151 (2004) “Paper and board—Determination of surface roughness/smoothness (air leak methods)—print-surf method” (ISO 8791-4 “Paper and board—Determination of roughness/smoothness (air leak methods)—Part 4: Print-surf method”).

The filter body 20 is, for example, a monolayer or multilayer paper sheet, or a structure having an opening (e.g., honeycomb structure).

The structure having an opening, particularly, a honeycomb structure, is preferred because visible light reaches the inside of the structure through the opening and the photocatalyst particles easily exhibit a photocatalytic function (that is, to improve deodorizing performance).

Examples of the honeycomb structure include an expandable honeycomb structure (see FIG. 1) and a corrugated board honeycomb structure (see FIG. 2). In FIG. 1 and FIG. 2, the reference symbol 20A indicates an opening of the structure.

The expandable honeycomb structure is, for example, a multilayer formed by stacking paper sheets that have been folded so as to form, when the paper sheets are stacked, openings of the expandable honeycomb structure.

The corrugated board structure is, for example, a multilayer formed by alternately stacking corrugated paper sheets, which have been folded in a corrugated shape, and flat paper sheets. The corrugated paper sheets are bonded to the flat paper sheets at the corrugation tops of the corrugated paper sheets.

Each paper sheet may be a monolayer paper sheet or a multilayer paper sheet.

To improve deodorizing performance, the average opening diameter of the openings of the honeycomb structure is preferably 0.5 mm or more and 15 mm or less, more preferably 1.0 mm or more and 10 mm or less, and still more preferably 2.0 mm or more and 5.0 mm or less.

The average opening diameter of the openings of the honeycomb structure is measured as described below.

The equivalent circular diameter of 10 openings is measured by observing the opening plane of the honeycomb structure. The arithmetic mean of the measured equivalent circular diameters is taken as the average opening diameter of the openings.

To improve deodorizing performance, the porosity of the opening plane of the honeycomb structure is preferably 20% or more and 80% or less, more preferably 30% or more and 70% or less, and still more preferably 35% or more and 60% or less.

The porosity of the opening plane of the honeycomb structure is measured as described below.

The total area of the openings is measured by observing the entire opening plane of the honeycomb structure. The proportion of the area of the openings relative to the area of the opening plane of the honeycomb structure is calculated and taken as the porosity of the opening plane of the honeycomb structure.

The filter body 20 may have any shape and may have a well-known shape, such as plate shape, cylindrical shape, hollow cylindrical shape, prismatic shape, or hollow prismatic shape.

Next, the material of the filter body 20 will be described.

The material of the filter body 20 is a paper material. The paper material contains a pulp fiber (including cellulose fiber) as a main component. The paper material may also contain various well-known additives, such as filler, as needed. The main component is a component present in the greatest proportion (% by volume) among all components.

Examples of the pulp fiber (including cellulose fiber) include chemical pulp, such as bleached hardwood kraft pulp, unbleached hardwood kraft pulp, bleached softwood kraft pulp, unbleached softwood kraft pulp, bleached hardwood sulfite pulp, unbleached hardwood sulfite pulp, bleached softwood sulfite pulp, and unbleached softwood sulfite pulp; and well-known pulp, such as pulp produced by chemically processing fiber raw materials, such as used paper, wood, cotton, hemp, and bast.

To improve deodorizing performance and improve deodorization maintainability, the average fiber diameter of the pulp fiber (including cellulose fiber) is preferably 5 μm or more and 70 μm or less, more preferably 10 μm or more and 50 μm or less, and still more preferably 10 μm or more and 30 μm or less.

To improve deodorizing performance and improve deodorization maintainability, the average fiber length of the pulp fiber (including cellulose fiber) is preferably 0.5 mm or more and 5 mm or less, more preferably 0.8 mm or more and 1.8 mm or less, and still more preferably 1.0 mm or more and 1.5 mm or less.

The average fiber diameter and the average fiber length of the pulp fiber are determined as follows: measuring the fiber diameter and the fiber length of 20 pulp fibers through electron microscopic observation; and calculating the arithmetic means thereof.

To improve deodorizing performance and improve deodorization maintainability, the basis weight of the paper material is preferably 30 g/m² or more and 210 g/m² or less, more preferably 40 g/m² or more and 150 g/m² or less, and still more preferably 50 g/m² or more and 100 g/m² or less.

The basis weight of the paper material is a value determined in accordance with JIS P 8124 (2011).

The paper material may be a sheet-shaped material. The paper material may be, for example, a line-shaped paper material or strip-shaped paper material. The line-shaped paper material or strip-shaped paper material may be, for example, woven or knitted to form a sheet-shaped material. The sheet-shaped material can be, for example, used as the paper sheet described above.

### Photocatalyst Particles

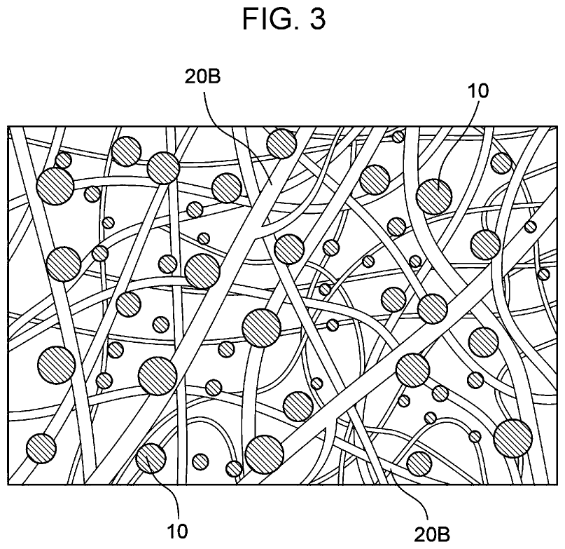

The photocatalyst particles 10 are attached to, for example, the filter body 20 (specifically, the surfaces of the pulp fibers of the paper that constitutes the surface layer of the filter body 20) (see FIG. 3). In FIG. 3, the reference symbol 20B indicates a pulp fiber of the paper.

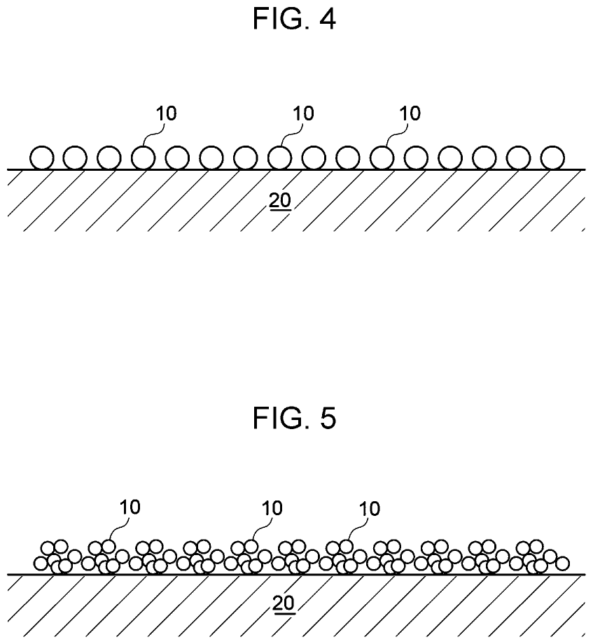

For example, when the photocatalyst particle 10 is a metatitanic acid particle or a titanium oxide particle, as illustrated in FIG. 4, the photocatalyst particles 10 in the form of primary particles may be attached to the filter body 20.

For example, when the photocatalyst particles 10 are titanium oxide aerogel particles and silica-titania composite aerogel particles, as illustrated in FIG. 5, the photocatalyst particles 10 in the form of aggregates having an aerogel structure may be attached to the filter body 20.

The term “aerogel structure” refers to the structure of aggregated primary particles forming a porous structure and indicates an internally three-dimensional mesh-like fine structure having a cluster structure formed by assembled particle-like materials with a nanometer order size.

The photocatalyst particles 10 will be described below in detail. The description will be given without reference numerals.

The photocatalyst particles have absorption at wavelengths of 450 nm and 750 nm in the visible absorption spectrum. Accordingly, the photocatalyst particles have a high photocatalytic function driven by visible light.

Specifically, the photocatalyst particle is a particle having the surface to which a metal compound having a metal atom and a hydrocarbon group is bonded via an oxygen atom.

The particle having the surface to which a metal compound having a metal atom and a hydrocarbon group is bonded via an oxygen atom is prepared as follows: for example, treating the surfaces of untreated particles (e.g., untreated metatitanic acid particles, untreated titanium oxide particles, untreated titanium oxide aerogel particles, and untreated silica-titania composite aerogel particles) with a metal compound having a hydrocarbon group; and oxidizing at least part of the hydrocarbon group by a heat treatment into C—O bonds or C═O bonds. Although the detailed mechanism is unclear, the surface of the particle exhibits light absorption at wavelengths of 450 nm and 750 nm due to the presence of the structure formed, on the surface of the particle, by sequentially bonding an organometallic compound having appropriately oxidized carbon atoms, an oxygen atom, and a titanium atom (or silicon atom) to each other through covalent bonds. Accordingly, the particle may exhibit a visible light-driven photocatalytic function (visible-light responsiveness).

Hereafter, the metal compound having a metal atom and a hydrocarbon group is also referred to simply as an “organometallic compound.”

The photocatalyst particle not only exhibits a high photocatalytic function even in the visible light region but also has the following features.

In general, untreated particles (e.g., untreated metatitanic acid particles, untreated titanium oxide particles, untreated titanium oxide aerogel particles, and untreated silica-titania composite aerogel particles) have high hydrophilicity and high aggregability and thus tend to have poor dispersion and adhesion with respect to the filter body.

When the surfaces of the photocatalyst particles have a hydrocarbon group derived from an organometallic compound, the photocatalyst particles have higher hydrophobicity and have improved dispersibility and adhesion with respect to the filter body. Thus, the photocatalyst particles are substantially uniformly attached to the surface of the filter body and unlikely to be detached from the filter body.

### Untreated Particles

Examples of particles (untreated particles) to be subjected to the surface treatment with an organometallic compound include untreated titanium compound particles. Examples of untreated titanium compound particles include untreated particles, such as untreated metatitanic acid particles, untreated titanium oxide particles, untreated titanium oxide aerogel particles, and untreated silica-titania composite aerogel particles. Among these particles, untreated metatitanic acid particles are preferred to improve deodorizing performance.

In other words, the photocatalyst particle may be at least one type of particle selected from the group consisting of a metatitanic acid particle, a titanium oxide particle, and a silica-titania composite particle. Metatitanic acid particles are preferred.

When photocatalyst particle in the form of aggregates having an aerogel structure are attached to the surface of the filter body, untreated titanium compound particles may be at least one type of particles selected from untreated titanium oxide aerogel particles and untreated silica-titania composite aerogel particles.

### Untreated Metatitanic Acid Particles

The untreated metatitanic acid particles refer to titanic acid particles of titanic acid hydrate TiO₂·nH₂O where n=1.

Examples of the method for producing untreated metatitanic acid particles include, but are not limited to, a chlorine method (gas phase method) and a sulfuric acid method (liquid phase method). A sulfuric acid method is preferred.

An example of the sulfuric acid method (liquid phase method) is as follows. First, ilmenite ore (FeTiO₃) or titanium slag, which is a raw material, is dissolved in concentrated sulfuric acid, and the iron component, which is an impurity, is separated in the form of iron sulfate (FeSO₄) to form titanium oxysulfate (TiOSO₄) (titanyl sulfate solution). Next, titanium oxysulfate (TiOSO₄) is hydrolyzed to produce untreated metatitanic acid [titanium oxyhydroxide (TiO(OH)₂)] particles.

### Untreated Titanium Oxide Particles

Examples of the untreated titanium oxide particles include brookite, anatase, and rutile titanium oxide particles. The titanium oxide particles may have a single-crystal structure, such as brookite, anatase, or rutile, or may have a mixed crystal structure where these crystals are present together.

Examples of the method for producing untreated titanium oxide particles include, but are not limited to, a chlorine method (gas phase method) and a sulfuric acid method (liquid phase method).

### Untreated Titanium Oxide Aerogel Particles

The untreated titanium oxide aerogel particles may be produced by a sol-gel method using a titanium alkoxide as a material.

The untreated titanium oxide aerogel particles may be formed of a hydrolysis-condensation product of the titanium alkoxide. Some of the alkoxy groups of the titanium alkoxide may remain unreacted in the particles.

The method for producing the untreated titanium oxide aerogel particles will be described below.

The method for producing the untreated titanium oxide aerogel particles may include at least the following steps (1) and (2).
(1) A step of making porous particles containing titanium oxide by a sol-gel method to prepare a dispersion containing the porous particles and a solvent (dispersion preparing step).
(2) A step of removing the solvent from the dispersion by using supercritical carbon dioxide (solvent removing step).

### (1) Dispersion Preparing Step

The dispersion preparing step involves, for example, causing the reactions (hydrolysis and condensation) of a titanium alkoxide, which is used as a material, to generate titanium oxide, forming a dispersion in which porous particles containing titanium oxide are dispersed in a solvent.

The dispersion preparing step is specifically, for example, the following step.

A titanium alkoxide is added to an alcohol and, under stirring, an acid aqueous solution is added dropwise thereto to cause the reaction of the titanium alkoxide and thus to generate titanium oxide, forming a dispersion (porous particle dispersion) in which porous particles containing titanium oxide are dispersed in the alcohol.

Here, the primary particle size of the porous particles can be controlled by the amount of the titanium alkoxide added in the dispersion preparing step. A larger amount of the titanium alkoxide added results in a smaller primary particle size of the porous particles. The mass ratio of the titanium alkoxide to the alcohol is preferably 0.04 or more and 0.65 or less, and more preferably 0.1 or more and 0.5 or less.

Examples of the titanium alkoxide used in the dispersion preparing step include tetraalkoxytitaniums, such as tetramethoxytitanium, tetraethoxytitanium, tetrapropoxytitanium, and tetrabutoxytitanium; alkoxy titanium chelates in which some of alkoxy groups are chelated, such as di-i-propoxy·bis(ethylacetate)titanium, and di-i-propoxy·bis(acetylacetonato)titanium. These titanium alkoxides may be used alone or in combination of two or more.

The titanium oxide aerogel particles may contain a small amount of a metal element other than titanium, such as silicon and aluminum. In this case, a tetraalkoxysilane, such as tetramethoxysilane, tetraethoxysilane, tetrapropoxysilane, or tetrabutoxysilane; an alkyltrialkoxysilane, such as methyltrimethoxysilane, methyltriethoxysilane, or ethyltriethoxysilane; an alkyldialkoxysilane, such as dimethyldimethoxysilane or dimethyldiethoxysilane; or an aluminum alkoxide, such as aluminum isopropoxide may be used. When the titanium oxide aerogel particles contain a silicon element, these substances can be used at an elemental ratio Si/Ti of silicon to titanium in the range from 0 to 0.05.

Examples of the alcohol used in the dispersion preparing step include methanol, ethanol, propanol, and butanol. These alcohols may be used alone or in combination of two or more.

Examples of the acid for the acid aqueous solution used in the dispersion preparing step include oxalic acid, acetic acid, hydrochloric acid, and nitric acid. The acid concentration of the acid aqueous solution is preferably 0.001% by mass or more and 1% by mass or less, and more preferably 0.005% by mass or more and 0.01% by mass or less.

The amount of the acid aqueous solution added dropwise in the dispersion preparing step is preferably 0.001 parts by mass or more and 0.1 parts by mass or less relative to 100 parts by mass of the titanium alkoxide.

The solid content of the porous particle dispersion prepared in the dispersion preparing step is preferably 1% by mass or more and 30% by mass or less.

### (2) Solvent Removing Step

The solvent removing step involves bringing supercritical carbon dioxide into contact with a dispersion containing porous particles and a solvent to remove the solvent. The solvent removing process using supercritical carbon dioxide is less likely to cause closing or clogging of pores of the porous particles than a solvent removing process performed by heating. When the solvent removing step is a step of removing the solvent by using supercritical carbon dioxide, titanium oxide aerogel particles having a BET specific surface area of 120 m²/g or more can be obtained.

The solvent removing step is specifically performed by, for example, using the following process.

To a sealed reactor, the porous particle dispersion is added, and liquefied carbon dioxide is next introduced. The sealed reactor is then heated, and the pressure in the sealed reactor is increased with a high-pressure pump to bring carbon dioxide in the sealed reactor into the supercritical state. Liquefied carbon dioxide is then introduced into the sealed reactor to discharge supercritical carbon dioxide from the sealed reactor, and supercritical carbon dioxide is circulated in the porous particle dispersion in the sealed reactor accordingly. During the circulation of supercritical carbon dioxide in the porous particle dispersion, the solvent is dissolved in supercritical carbon dioxide and removed together with supercritical carbon dioxide discharged from the sealed reactor.

The temperature and the pressure in the sealed reactor correspond to the temperature and the pressure at which carbon dioxide is converted into the supercritical state. Since the critical point of carbon dioxide is at 31.1° C./7.38 MPa, the temperature and the pressure are, for example, 50° C. or higher and 200° C. or lower/10 MPa or more and 30 MPa or less.

### Untreated Silica-Titania Composite Aerogel Particles

The untreated silica-titania composite aerogel particles contain a silica-titania composite, which is a silica-titanium composite oxide, as a main component (the greatest proportion of component among all particle components).

The elemental ratio Si/Ti of silicon to titanium in the untreated silica-titania composite aerogel particles is preferably more than 0 and 6 or less, more preferably 0.05 or more and 4 or less, and still more preferably 0.1 or more and 3 or less in order to exhibit a photocatalytic function in the visible light region.

The elemental ratio (Si/Ti) of silicon atoms to titanium atoms is determined on the basis of the elemental profiles for the silica-titania composite created by XPS qualitative analysis (wide scan analysis). Specifically, the elemental ratio (Si/Ti) is determined as described below.

The identification and quantitative determination of titanium atoms, silicon atoms, and carbon atoms are performed by qualitative analysis (wide scan analysis) involving etching the silica-titania composite in the depth direction from the surface by using an XPS analyzer under the following conditions. From the obtained data, the elemental profiles where the vertical axis indicates peak intensity and the horizontal axis indicates etching time are drawn for titanium atoms, silicon atoms, and carbon atoms. Each profile curve is divided into plural regions at inflection points. The region (region A described below) where the peak intensity of titanium atoms and the peak intensity of silicon atoms are substantially constant) is specified, and the elemental ratio Si/Ti in the region is determined.
XPS analyzer: available from Ulvac-Phi, Incorporated., Versa Probe II
X-ray source: monochromatic Al Kα rays
Acceleration voltage: 15 kV
X-ray beam diameter: 100 μm
Etching gun: argon ion beam
Etching output: 4 kV

In the untreated silica-titania composite aerogel particles, the total amount of the silica component and the titania component is preferably 80% by mass or more, more preferably 90% by mass or more, and still more preferably 95% by mass or more relative to the total mass of the composite.

The untreated silica-titania composite aerogel particles may contain base particles having an elemental ratio Si/Ti of silicon to titanium of more than 0 and 6 or less, and a titania layer (layer made of titania) present on the surfaces of the base particles. In other words, the untreated silica-titania composite aerogel particles may be particles having a titania layer in its surface layer. The use of these particles may improve the photocatalytic function.

The method for producing the untreated silica-titania composite aerogel particles may be a sol-gel method using an alkoxy silane and a titanium alkoxide as materials.

The untreated silica-titania composite aerogel particles may be formed of a hydrolysis-condensation product of an alkoxy silane and a titanium alkoxide. However, some of hydrocarbon groups, such as alkoxy groups, of the alkoxy silane or the titanium alkoxide may remain unreacted in the composite.

The method for producing the untreated silica-titania composite aerogel particles will be described below.

The method for producing the untreated silica-titania composite aerogel particles may include at least the following steps (1′) and (2′).
(1′) A step of making porous particles containing a silica-titania composite by a sol-gel method to prepare a dispersion containing the porous particles and a solvent (dispersion preparing step).
(2′) A step of removing the solvent from the dispersion by using supercritical carbon dioxide (solvent removing step).
(1′) Dispersion Preparing Step

The dispersion preparing step involves, for example, causing the reactions (hydrolysis and condensation) of an alkoxy silane and a titanium alkoxide, which are used as materials, to generate a silica-titania composite, forming a dispersion in which porous particles containing the silica-titania composite are dispersed in a solvent. Here, the porous particles may be aggregated particles generated by aggregating primary particles that contain the silica-titania composite and form a porous structure.

The dispersion preparing step is specifically, for example, the following step.

An alkoxy silane and a titanium alkoxide are added to an alcohol and, under stirring, an acid aqueous solution is added dropwise thereto to cause the reaction of the alkoxy silane and the titanium alkoxide and thus to generate a silica-titania composite, forming a dispersion (porous particle dispersion) in which porous particles containing the silica-titania composite are dispersed in the alcohol.

The elemental ratio Si/Ti of silicon and titanium in the untreated silica-titania composite aerogel particles can be controlled by adjusting the mixing ratio of the alkoxy silane and the titanium alkoxide in the dispersion preparing step.

The particle size of the primary particles forming the untreated silica-titania aerogel particles and the particle size of the untreated silica-titania aerogel particles can be controlled by the total amount of the alkoxy silane and the titanium alkoxide relative to the amount of the alcohol in the dispersion preparing step. As the total amount relative to the amount of the alcohol increases, the particle size of the primary particles forming the untreated silica-titania composite aerogel particles decreases, and the particle size of the untreated silica-titania composite aerogel particles increases. The total amount of the alkoxy silane and the titanium alkoxide is preferably 4 parts by mass or more and 250 parts by mass or less, and more preferably 10 parts by mass or more and 50 parts by mass or less relative to 100 parts by mass of the alcohol.

Examples of the alkoxy silane used in the dispersion preparing step include tetraalkoxysilanes, such as tetramethoxysilane, tetraethoxysilane, tetrapropoxysilane, and tetrabutoxysilane; alkyltrialkoxysilanes, such as methyltrimethoxysilane, methyltriethoxysilane, and ethyltriethoxysilane; and alkyldialkoxysilanes, such as dimethyldimethoxysilane and dimethyldiethoxysilane. These alkoxy silanes may be used alone or in combination of two or more.

Examples of the titanium alkoxide used in the dispersion preparing step include tetraalkoxytitaniums, such as tetramethoxytitanium, tetraethoxytitanium, tetrapropoxytitanium, and tetrabutoxytitanium; and alkoxy titanium chelates in which some of alkoxy groups are chelated, such as di-i-propoxy·bis(ethylacetoacetate)titanium, and di-i-propoxy·bis(acetylacetonato)titanium. These titanium alkoxides may be used alone or in combination of two or more.

Examples of the alcohol used in the dispersion preparing step include methanol, ethanol, propanol, and butanol. These alcohols may be used alone or in combination of two or more.

Examples of the acid for the acid aqueous solution used in the dispersion preparing step include oxalic acid, acetic acid, hydrochloric acid, and nitric acid. The acid concentration of the acid aqueous solution is preferably 0.001% by mass or more and 1% by mass or less, and more preferably 0.005% by mass or more and 0.01% by mass or less.

The amount of the acid aqueous solution added dropwise in the dispersion preparing step is preferably 0.001 parts by mass or more and 0.1 parts by mass or less relative to 100 parts by mass of the total amount of the alkoxy silane and the titanium alkoxide.

The solid content of the porous particle dispersion prepared in the dispersion preparing step is preferably 1% by mass or more and 30% by mass or less.

### (2′) Solvent Removing Step

The solvent removing step involves bringing supercritical carbon dioxide into contact with a dispersion containing porous particles and a solvent to remove the solvent. The solvent removing process using supercritical carbon dioxide is less likely to cause closing or clogging of pores of the porous particles (in particular, aggregated particles generated by aggregating primary particles forming a porous structure) than a solvent removing process performed by heating. When the solvent removing step is a step of removing the solvent by using supercritical carbon dioxide, untreated silica-titania composite aerogel particles having a BET specific surface area of 200 m²/g or more can be obtained.

The solvent removing step is specifically performed by, for example, using the following process.

To a sealed reactor, the porous particle dispersion is added, and liquefied carbon dioxide is next introduced. The sealed reactor is then heated, and the pressure in the sealed reactor is increased with a high-pressure pump to bring carbon dioxide in the sealed reactor into the supercritical state. Liquefied carbon dioxide is then introduced into the sealed reactor to discharge supercritical carbon dioxide from the sealed reactor, and supercritical carbon dioxide is circulated in the porous particle dispersion in the sealed reactor accordingly. During the circulation of supercritical carbon dioxide in the porous particle dispersion, the solvent is dissolved in supercritical carbon dioxide and removed together with supercritical carbon dioxide discharged from the sealed reactor.

The temperature and the pressure in the sealed reactor correspond to the temperature and the pressure at which carbon dioxide is converted into the supercritical state. Since the critical point of carbon dioxide is at 31.1° C./7.38 MPa, the temperature and the pressure are, for example, 50° C. or higher and 200° C. or lower/10 MPa or more and 30 MPa or less.

When particles having a titania layer in its surface layer are produced as untreated silica-titania composite aerogel particles, the dispersion preparing step (1′) may involve the following operations (i) and (ii).
(i) An alkoxy silane and a titanium alkoxide are added to an alcohol and, under stirring, an acid aqueous solution is added dropwise thereto to cause the reaction of the alkoxy silane and the titanium alkoxide and thus to generate a silica-titania composite, forming a dispersion (first dispersion) in which base particles containing the silica-titania composite are dispersed in the alcohol.
(ii) A mixture formed by mixing an alcohol and a titanium alkoxide is added dropwise to the first dispersion under stirring to cause the reaction of the base particles and the titanium alkoxide and thus to generate porous particles having an intermediate layer on the surfaces of the base particles, forming a dispersion (second dispersion) in which the porous particles are dispersed in the alcohol.

### Organometallic Compound

The organometallic compound is a metal compound having a metal atom and a hydrocarbon group.

In order that the photocatalyst particles easily exhibit visible-light responsiveness, the organometallic compound may be a metal compound having a metal atom, a carbon atom, a hydrogen atom, and an oxygen atom.

In order that the photocatalyst particles easily exhibit visible-light responsiveness, the organometallic compound may be bonded to the surfaces of the particles via an oxygen atom O directly bonded to the metal atom M in the organometallic compound, that is, may be bonded to the surfaces of the particles through covalent bonds M—O—Ti (or M—O—Si).

In order that the photocatalyst particles easily exhibit visible-light responsiveness, the organometallic compound may be an organometallic compound having a metal atom M and a hydrocarbon group directly bonded to the metal atom M. The organometallic compound may be bonded to the surface of the particle via an oxygen atom O directly bonded to the metal atom M in the organometallic compound. In other words, the surface of the particle may have a structure (hydrocarbon group-M—O—Ti (or hydrocarbon group-M—O—Si)) formed by sequentially bonding a hydrocarbon group, a metal atom M, an oxygen atom O, and a titanium atom Ti to each other through covalent bonds, in order that the photocatalyst particles easily exhibit visible-light responsiveness.

When the organometallic compound has plural hydrocarbon groups, at least one of the hydrocarbon groups may be directly bonded to the metal atom in the organometallic compound.

The chemical bonding state between atoms in the organometallic compound can be determined by high-resolution X-ray Photoelectron Spectroscopy (XPS) analysis (narrow scan analysis).

The metal atom M of the organometallic compound is preferably silicon, aluminum, or titanium, more preferably silicon or aluminum, and still more preferably silicon.

Examples of the hydrocarbon group of the organometallic compound include saturated or unsaturated aliphatic hydrocarbon groups having 1 or more and 40 or less carbon atoms (preferably 1 or more and 20 or less carbon atoms, more preferably 1 or more and 18 or less carbon atoms, still more preferably 4 or more and 12 or less carbon atoms, and yet still more preferably 4 or more and 10 or less carbon atoms), and aromatic hydrocarbon groups having 6 or more and 27 or less carbon atoms (preferably 6 or more and 20 or less carbon atoms, more preferably 6 or more and 18 or less carbon atoms, still more preferably 6 or more and 12 or less carbon atoms, and yet still more preferably 6 or more and 10 or less carbon atoms).

To exhibit a high photocatalytic function and improve dispersibility, the hydrocarbon group of the organometallic compound is preferably an aliphatic hydrocarbon group, more preferably a saturated aliphatic hydrocarbon group, and still more preferably an alkyl group. The aliphatic hydrocarbon group may be a straight-chain, branched-chain, or cyclic hydrocarbon group, but preferably a straight-chain or branched-chain hydrocarbon group in view of dispersibility. The number of carbons in the aliphatic hydrocarbon group is preferably 1 or more and 20 or less, more preferably 1 or more and 18 or less, still more preferably 4 or more and 12 or less, and yet still more preferably 4 or more and 10 or less.

The organometallic compound may be a silane compound having a hydrocarbon group. Examples of the silane compound having a hydrocarbon group include a chlorosilane compound and alkoxy silane compounds.

To exhibit a high photocatalytic function and improve dispersibility, the silane compound having a hydrocarbon group may be a compound represented by formula (1) of R¹ₙSiR²ₘ.

In formula (1) of R¹ₙSiR²ₘ, R¹ represents a saturated or unsaturated aliphatic hydrocarbon group having 1 or more and 20 or less carbon atoms or an aromatic hydrocarbon group having 6 or more and 20 or less carbon atoms, R2 represents a halogen atom or an alkoxy group, n represents an integer of 1 or more and 3 or less, and m represents an integer of 1 or more and 3 or less, wherein n+m=4. When n is an integer of 2 or 3, plural R¹'s may be the same group or different groups. When m is an integer of 2 or 3, plural R²'s may be the same group or different groups.

The aliphatic hydrocarbon group represented by R¹ may be a straight-chain, branched-chain, or cyclic hydrocarbon group, but preferably a straight-chain or branched-chain hydrocarbon group in view of dispersibility. To exhibit a high photocatalytic function and improve dispersibility, the number of carbons in the aliphatic hydrocarbon group is preferably 1 or more and 20 or less, more preferably 1 or more and 18 or less, still more preferably 4 or more and 12 or less, and yet still more preferably 4 or more and 10 or less. The aliphatic hydrocarbon group may be saturated or unsaturated. To exhibit a high photocatalytic function and improve dispersibility, the aliphatic hydrocarbon group is preferably a saturated aliphatic hydrocarbon group, and more preferably an alkyl group.

Examples of the saturated aliphatic hydrocarbon group include straight-chain alkyl groups (e.g., a methyl group, an ethyl group, a propyl group, a butyl group, a pentyl group, a hexyl group, a heptyl group, an octyl group, a nonyl group, a decyl group, a dodecyl group, a hexadecyl group, and an icosyl group), branched-chain alkyl groups (e.g., an isopropyl group, an isobutyl group, an isopentyl group, a neopentyl group, a 2-ethylhexyl group, a tertiary butyl group, a tertiary pentyl group, and an isopentadecyl group), and cyclic alkyl groups (e.g., a cyclopropyl group, a cyclopentyl group, a cyclohexyl group, a cycloheptyl group, a cyclooctyl group, a tricyclodecyl group, a norbornyl group, and an adamantyl group).

Examples of the unsaturated aliphatic hydrocarbon group include alkenyl groups (e.g., a vinyl group (ethenyl group), a 1-propenyl group, a 2-propenyl group, a 2-butenyl group, a 1-butenyl group, a 1-hexenyl group, a 2-dodecenyl group, and a pentenyl group), and alkynyl groups (e.g., an ethynyl group, a 1-propynyl group, a 2-propynyl group, a 1-butynyl group, a 3-hexynyl group, and a 2-dodecynyl group).

Aliphatic hydrocarbon groups include substituted aliphatic hydrocarbon groups. Examples of the substituent that may substitute aliphatic hydrocarbon groups include halogen atoms, an epoxy group, a glycidyl group, a glycidoxy group, a mercapto group, a methacryloyl group, and an acryloyl group.

The aromatic hydrocarbon group represented by R¹ preferably has 6 or more and 20 or less carbon atoms, more preferably has 6 or more and 18 or less carbon atoms, still more preferably has 6 or more and 12 or less carbon atoms, and yet still more preferably has 6 or more and 10 or less carbon atoms.

Examples of the aromatic hydrocarbon group include a phenylene group, a biphenylene group, a terphenylene group, a naphthalene group, and an anthracene group.

Aromatic hydrocarbon groups include substituted aromatic hydrocarbon groups. Examples of the substituent that may substitute aromatic hydrocarbon groups include halogen atoms, an epoxy group, a glycidyl group, a glycidoxy group, a mercapto group, a methacryloyl group, and an acryloyl group.

Examples of the halogen atom represented by R² include a fluorine atom, a chlorine atom, a bromine atom, and an iodine atom. The halogen atom is preferably a chlorine atom, a bromine atom, or an iodine atom.

Examples of the alkoxy group represented by R² include alkoxy groups having 1 or more and 10 or less carbon atoms (preferably 1 or more and 8 or less carbon atoms, and more preferably 3 or more and 8 or less carbon atoms). Examples of the alkoxy group include a methoxy group, an ethoxy group, an isopropoxy group, a t-butoxy group, an n-butoxy group, an n-hexyloxy group, a 2-ethylhexyloxy group, and a 3,5,5-trimethylhexyloxy group. Alkoxy groups include substituted alkoxy groups. Examples of the substituent that may substitute alkoxy groups include halogen atoms, a hydroxyl group, amino groups, alkoxy groups, amide groups, and carbonyl groups.

The compound represented by formula (1) of R¹ₙSiR²ₘ is preferably a compound where R¹ is a saturated aliphatic hydrocarbon group, in order to exhibit a high photocatalytic function and improve dispersibility. In particular, the compound represented by formula (1) of R¹ₙSiR²ₘ is more preferably a compound where R¹ is a saturated aliphatic hydrocarbon group having 1 or more and 20 or less carbon atoms, R² is a halogen atom or an alkoxy group, n is an integer of 1 or more and 3 or less, and m is an integer of 1 or more and 3 or less, wherein n+m=4.

Examples of the compound represented by formula (1) of R¹ₙSiR²ₘ, include silane compounds, such as vinyltrimethoxysilane, methyltrimethoxysilane, ethyltrimethoxysilane, propyltrimethoxysilane, butyltrimethoxysilane, hexyltrimethoxysilane, n-octyltrimethoxysilane, decyltrimethoxysilane, dodecyltrimethoxysilane, vinyltriethoxysilane, methyltriethoxysilane, ethyltriethoxysilane, butyltriethoxysilane, hexyltriethoxysilane, decyltriethoxysilane, dodecyltriethoxysilane, phenyltrimethoxysilane, o-methylphenyltrimethoxysilane, p-methylphenyltrimethoxysilane, phenyltriethoxysilane, benzyltriethoxysilane, decyltrichlorosilane, and phenyltrichlorosilane (compounds where n=1 and m=3);

dimethyldimethoxysilane, dimethyldiethoxysilane, methylvinyldimethoxysilane, methylvinyldiethoxysilane, diphenyldimethoxysilane, diphenyldiethoxysilane, dimethyldichlorosilane, and dichlorodiphenylsilane (compounds where n=2 and m=2);

trimethylmethoxysilane, trimethylethoxysilane, trimethylchlorosilane, decyldimethylchlorosilane, triphenylchlorosilane (compounds where n=3 and m=1); and

3-glycidoxypropyltrimethoxysilane, γ-methacryloxypropyltrimethoxysilane, γ-mercaptopropyltrimethoxysilane, γ-chloropropyltrimethoxysilane, γ-aminopropyltrimethoxysilane, γ-aminopropyltriethoxysilane, γ-(2-aminoethyl)aminopropyltrimethoxysilane, γ-(2-aminoethyl)aminopropylmethyldimethoxysilane, γ-glycidyloxypropylmethyldimethoxysilane (compounds where R¹ is a substituted aliphatic hydrocarbon group or a substituted aromatic hydrocarbon group). The silane compound may be used alone or in combination of two or more.

To exhibit a high photocatalytic function and improve dispersibility, the hydrocarbon group in the silane compound represented by formula (1) is preferably an aliphatic hydrocarbon group, more preferably a saturated aliphatic hydrocarbon group, and still more preferably an alkyl group. To exhibit a high photocatalytic function and improve dispersibility, the hydrocarbon group in the silane compound is preferably a saturated aliphatic hydrocarbon group having 1 or more and 20 or less carbon atoms, more preferably a saturated aliphatic hydrocarbon group having 1 or more and 18 or less carbon atoms, still more preferably a saturated aliphatic hydrocarbon group having 4 or more and 12 or less carbon atoms, and yet still more preferably a saturated aliphatic hydrocarbon group having 4 or more and 10 or less carbon atoms.

Examples of organometallic compounds where the metal atom is aluminum include alkyl aluminates, such as triethoxyaluminum, tri-i-propoxyaluminum, and tri-sec-butoxyaluminum; aluminum chelates, such as di-i-propoxymono-sec-butoxyaluminum and di-i-propoxyaluminum·ethylacetoacetate; and aluminate coupling agents, such as acetoalkoxyaluminum diisopropylate.

Examples of organometallic compounds where the metal atom is titanium include titanate coupling agents, such as isopropyl triisostearoyl titanate, tetraoctyl bis(ditridecylphosphite)titanate, and bis(dioctylpyrophosphate)oxyacetate titanate; and titanium chelates, such as di-i-propoxy bis(ethylacetoacetate)titanium, di-i-propoxy bis(acetylacetonate)titanium, di-i-propoxy bis(triethanolaminate)titanium, di-i-propoxy titanium diacetate, and di-i-propoxy titanium dipropionate.

The organometallic compound may be used alone or in combination of two or more.

### Method for Producing Photocatalyst Particles

The method for producing the photocatalyst particles is not limited. For example, the photocatalyst particles are produced by treating the surfaces of untreated particles with an organometallic compound.

An exemplary embodiment of the method for producing the photocatalyst particles will be described below.

The method for producing the photocatalyst particles may include, for example, (a) a step of treating the surfaces of untreated particles with an organometallic compound, and (b) a step of heating the particles during or after the step of treating the surfaces of untreated particles.

### (a) Surface Treatment Step

Examples of the method for treating the surfaces of untreated particles with an organometallic compound include, but are not limited to, a method of bringing the organometallic compound itself into direct contact with the untreated particles; and a method of bringing a treatment solution, which is prepared by dissolving the organometallic compound in a solvent, into contact with the untreated particles. Specific examples include a method of adding, under stirring, the organometallic compound itself or the treatment solution to a dispersion prepared by dispersing the untreated particles in a solvent; and a method of adding (e.g., by dropping or spraying) the organometallic compound itself or the treatment solution to the untreated particles in the state of being fluidized, for example, by stirring with a HENSCHEL MIXER or the like. In these methods, a reactive group (e.g., a hydrolyzable group, such as a halogen group or an alkoxy group) in the organometallic compound reacts with a hydroxyl group on the surfaces of the untreated particles, and the untreated particles undergo surface treatment accordingly.

The surface treatment step can be performed in the air or a nitrogen atmosphere. In the case of treating the surfaces of titanium oxide aerogel particles or silica-titania composite aerogel particles, which are used as untreated particles, the surface treatment step may be performed in supercritical carbon dioxide. In this process, the organometallic compound reaches deep into the pores of the porous particles, and the surface treatment is achieved deeply into the pores of the porous particles. The surface treatment may thus be performed in supercritical carbon dioxide.

The surface treatment step performed in supercritical carbon dioxide involves, for example, mixing an organometallic compound and a porous body in supercritical carbon dioxide under stirring to cause them to react with each other. Alternatively, the surface treatment step involves, for example, preparing a treatment solution by mixing an organometallic compound and a solvent, and mixing a porous body and the treatment solution in supercritical carbon dioxide under stirring. To increase the specific surface area with the pore structure of the porous body maintained, the organometallic compound may be placed in supercritical carbon dioxide after completion of the solvent removing step, causing the reaction of the organometallic compound with the surface of the porous body in supercritical carbon dioxide.

Examples of the solvent used to dissolve the organometallic compound include organic solvents (e.g., hydrocarbon solvents, ester solvents, ether solvents, halogenated solvents, alcohol solvents), water, and mixed solvents thereof. Examples of hydrocarbon solvents include toluene, benzene, xylene, hexane, octane, hexadecane, and cyclohexane. Examples of ester solvents include methyl acetate, ethyl acetate, isopropyl acetate, and amyl acetate. Examples of ester solvents include dibutyl ether and dibenzyl ether. Examples of halogenated solvents include 1,1-dichloro-1-fluoroethane, 1,1-dichloro-2,2,2-trifluoroethane, 1,1-dichloro-2,2,3,3,3-pentafluoropropane, chloroform, dichloroethane, and carbon tetrachloride. Examples of alcohol solvents include methanol, ethanol, and i-propyl alcohol. Examples of water include tap water, distilled water, and pure water. In addition to these solvents, solvents, such as dimethylformamide, dimethylacetamide, dimethyl sulfoxide, acetic acid, and sulfuric acid, may be used.

In the treatment solution prepared by dissolving the organometallic compound in the solvent, the concentration of the organometallic compound is preferably 0.05 mol/L or more and 500 mol/L or less, and more preferably 0.5 mol/L or more and 10 mol/L or less.

To exhibit a high photocatalytic function and improve dispersibility, the conditions for the surface treatment of the particles with the organometallic compound may be as follows. The surfaces of the untreated particles may be treated with 10% by mass or more and 100% by mass or less (preferably 20% by mass or more and 75% by mass or less, more preferably 25% by mass or more and 50% by mass or less) of the organometallic compound relative to the untreated particles. When the amount of the organometallic compound is 10% by mass or more, a high photocatalytic function tends to be exhibited even in the visible light region, and the dispersibility tends to be high. When the amount of the organometallic compound is 100% by mass or less, an excessive amount of the metal derived from the organometallic compound is unlikely to be present on the surfaces of the particles, and the deterioration of the photocatalytic function due to an excessive amount of the metal is unlikely to occur.

The temperature of the surface treatment of the untreated particles with the organometallic compound is preferably 15° C. or higher and 150° C. or lower, and more preferably 20° C. or higher and 100° C. or lower. The time of the surface treatment is preferably 10 minutes or longer and 120 minutes or shorter, and more preferably 30 minutes or longer and 90 minutes or shorter.

In the case of the surface treatment in supercritical carbon dioxide, the temperature and the pressure in the surface treatment step are the temperature and the pressure at which carbon dioxide is converted into the supercritical state. For example, the surface treatment step is performed in an atmosphere at a temperature of 50° C. or higher and 200° C. or lower and at a pressure of 10 MPa or higher and 30 MPa or lower. The reaction time is preferably 10 minutes or longer and 24 hours or shorter, more preferably 20 minutes or longer and 120 minutes or shorter, and still more preferably 30 minutes or longer and 90 minutes or shorter.

After the surface treatment of the untreated particles with the organometallic compound, a drying treatment may be performed. The drying treatment is not limited to any particular method and performed by a known drying method, such as a vacuum-drying method or a spray-drying method. The drying temperature may be 20° C. or higher and 150° C. or lower.

In the case of the surface treatment in supercritical carbon dioxide, a step of removing the solvent from the dispersion containing porous particles by using supercritical carbon dioxide is preferred, and a step of removing the solvent by circulating supercritical carbon dioxide in the dispersion after completion of the surface treatment step is more preferred.

### (b) Heat Treatment Step

A heat treatment is performed during the step of treating the surfaces of the untreated particles or after the step of treating the surfaces of the untreated particles.

A heat treatment can be performed during the surface treatment of the untreated particles with the organometallic compound; during the drying treatment after the surface treatment; or separately after the drying treatment. To cause a sufficient reaction between the particles and the organometallic compound before the heat treatment, the heat treatment is preferably performed during the drying treatment after the surface treatment, or separately after the drying treatment. To appropriately perform the drying treatment, the heat treatment is more preferably performed separately after the drying treatment.

To exhibit a high photocatalytic function and improve dispersibility, the temperature of the heat treatment is preferably 180° C. or higher and 500° C. or lower, more preferably 200° C. or higher and 450° C. or lower, and still more preferably 250° C. or higher and 400° C. or lower. To exhibit a high photocatalytic function and improve dispersibility, the time of the heat treatment is preferably 10 minutes or longer and 300 minutes or shorter, and more preferably 30 minutes or longer and 120 minutes or shorter. When the heat treatment is performed during the step of treating the surfaces of the untreated particles, the heat treatment may be performed at the above-described heat treatment temperature after the organometallic compound is caused to sufficiently react with the particles at the above-described surface treatment temperature. When the heat treatment is performed in the drying treatment after the surface treatment, the temperature of the drying treatment corresponds to the temperature of the heat treatment.

When the temperature of the heat treatment is 180° C. or higher and 500° C. or lower, particles that exhibit a high photocatalytic function even in the visible light region are obtained efficiently. The heat treatment at 180° C. or higher and 500° C. or lower may appropriately oxidize the hydrocarbon group derived from the metal compound present on the surfaces of the particles and may change some of C—C bonds or C═C bonds into C—O bonds or C═O bonds.

The heat treatment may be performed in an atmosphere with an oxygen concentration (% by volume) of 1% or more and 21% or less. The heat treatment in this oxygen atmosphere can appropriately and efficiently oxidize the hydrocarbon group derived from the metal compound present on the surfaces of the particles. The oxygen concentration (% by volume) is more preferably 3% or more and 21% or less and still more preferably 5% or more and 21% or less.

Examples of the method for the heat treatment include, but are not limited to, known heating methods, such as heating in an electric furnace, a firing furnace (e.g., Roller hearth kiln, Shuttle kiln), and a radiation heating furnace; and heating with a laser beam, infrared rays, UV rays, a microwave, and the like.

The photocatalyst particles are appropriately produced through the above-described steps.

### Characteristics of Photocatalyst Particles

The photocatalyst particles have absorption at wavelengths of 450 nm and 750 nm in the visible absorption spectrum.

In order that the photocatalyst particles exhibit a high photocatalytic function even in the visible light region, the photocatalyst particles preferably have absorption at wavelengths of 450 nm, 600 nm, and 750 nm in the visible absorption spectrum, more preferably have absorption in the entire wavelength range of 450 nm or more and 750 nm or less in the visible absorption spectrum, and still more preferably have absorption in the entire wavelength range of 400 nm or more and 800 nm or less in the visible absorption spectrum.

In order that the photocatalyst particles exhibit a high photocatalytic function even in the visible light region, assuming that the absorbance at a wavelength of 350 nm is 1, the absorbance at a wavelength of 450 nm is 0.02 or more, preferably 0.1 or more, more preferably 0.2 or more, and still more preferably 0.3 or more, the absorbance at a wavelength of 600 nm is 0.02 or more, preferably 0.1 or more, and more preferably 0.2 or more, and the absorbance at a wavelength of 750 nm is 0.02 or more, preferably 0.05 or more, and more preferably 0.1 or more, in the ultraviolet-visible absorption spectrum.

The ultraviolet-visible absorption spectrum of the photocatalyst particles is obtained by the following method. The particles targeted for measurement are dispersed in tetrahydrofuran, and the dispersion is then applied to a glass substrate and dried at 24° C. in the air. Using a spectrophotometer (e.g., U-4100 available from Hitachi High-Technologies Corporation, scanning speed: 600 nm, slit width: 2 nm, sampling interval: 1 nm), a diffuse reflectance spectrum in the wavelength range from 200 nm to 900 nm is measured in a diffuse reflectance configuration. From the diffuse reflectance spectrum, the absorbance at given wavelengths is theoretically determined by Kubelka-Munk conversion to obtain an ultraviolet-visible absorption spectrum.

The photocatalyst particle may have an absorption peak in the wavenumber range of 2700 cm⁻¹ or more and 3000 cm⁻¹ or less in the infrared absorption spectrum.

Specifically, for example, the photocatalyst particle may have at least one absorption peak in the wavenumber range of 2700 cm⁻¹ or more and 3000 cm⁻¹ or less in the infrared absorption spectrum. The expression “having an absorption peak” means having absorption with an absorption intensity (absorbance) of 0.022 (transmittance 5%) or more.

The infrared absorption spectrum of the photocatalyst particles is measured by the following method. First, titanium oxide particles targeted for measurement are prepared as a test sample by using the KBr pellet method. The infrared absorption spectrum of the test sample in the wavenumber range of 500 cm⁻¹ or more and 4,000 cm⁻¹ or less is then determined with an infrared spectrophotometer (FT-IR-410 available from JASCO Corporation) under the conditions of an integration number of 300 times and a resolution of 4 cm⁻¹.

The average particle size of the photocatalyst particles is preferably 0.01 μm or more and 0.5 μm or less, more preferably 0.02 μm or more and 0.15 μm or less, and still more preferably 0.02 μm or more and 0.1 μm or less. When the average particle size of the photocatalyst particles is 0.01 μm or more, the photocatalyst particles are unlikely to aggregate and thus tend to have a high photocatalytic function. When the average particle size of the photocatalyst particles is 0.5 μm or less, the ratio of the specific surface area to the amount is large, and the photocatalyst particles tend to have a high photocatalytic function. When the average particle size of the photocatalyst particles is in the above-described range, the photocatalyst particles tend to exhibit a high photocatalytic function in the visible light region.

The average particle size of the photocatalyst particles refers to, when the photocatalyst particles are aerogel particles, the average particle size of the primary particles forming the aerogel particles.

To improve deodorization maintainability, the ratio (average particle size/surface roughness) of the average particle size of the photocatalyst particles to the surface roughness of the filter body is preferably 0.001 or more and 0.5 or less, more preferably 0.005 or more and 0.1 or less, and still more preferably 0.01 or more and 0.05 or less.

To improve deodorization maintainability, the ratio (average particle size/fiber diameter) of the average particle size of the photocatalyst particles and the fiber diameter of the paper filter body (i.e., the average fiber diameter of the pulp fiber) is preferably 0.0001 or more and 0.1 or less, more preferably 0.001 or more and 0.05 or less, and still more preferably 0.005 or more and 0.01 or less.

The average particle size of the photocatalyst particles is the average particle size of the primary particles (average primary particle size) and is measured as described below.

While the photocatalyst particles are held on (attached to) the filter body, the image of the photocatalyst particles is captured by observation under a scanning electron microscope (S-4100 available from Hitachi, Ltd.). In this case, the image is captured with the scanning electron microscope at a magnification adjusted so as to perform image analysis on plural primary particles. The captured image is loaded into an image analyzer (LUZEXIII available from Nireco Corporation). The area of each particle is determined by image analysis of the primary particles, and the equivalent circular diameter (μm) is calculated from the area. The mean of the equivalent circular diameter is taken as an average primary particle size (μm). The average primary particle size is determined by analyzing about 10 to 50 primary particles.

When the photocatalyst particles are silica-titania composite aerogel particles, the photocatalyst particles may be particles formed by treating, with the organometallic compound, the surfaces of untreated silica-titania composite aerogel particles having a titania layer in its surface layer.

Specifically, these particles contain base particles (e.g., base particles having an elemental ratio Si/Ti of silicon to titanium of more than 0 and 6 or less), a titania layer (hereinafter also referred to as an “intermediate layer”) present on the surfaces of the base particles, and a layer formed, on the surface of the titania layer, by bonding a metal compound having a metal atom and a hydrocarbon group to the surface via an oxygen atom (i.e., a layer containing a metal compound having a metal atom and a hydrocarbon group, hereinafter also referred to as a “surface layer”).

The following method can confirm that the silica-titania composite aerogel particles have the above-described layers. The following method can also confirm that particles other than the silica-titania composite aerogel particles have a surface layer.

The XPS qualitative analysis (wide scan analysis) is performed by etching the silica-titania composite aerogel particles with a rare gas ion in the depth direction from the surface layer to identify and quantitatively determine at least titanium, silicon, and carbon. From the obtained data, the elemental profiles where the vertical axis indicates peak intensity and the horizontal axis indicates etching time are drawn for at least titanium, silicon, and carbon. Each profile curve is divided into plural regions at inflection points, and the following regions are specified: a region that reflects the elemental composition of the base particles, a region that reflects the elemental composition of the intermediate layer, and a region that reflects the elemental composition of the surface layer. When the elemental profiles include a region that reflects the elemental composition of the intermediate layer, the silica-titania composite aerogel particles are determined to have the intermediate layer. When the elemental profiles include a region that reflects the elemental composition of the surface layer, the silica-titania composite aerogel particles are determined to have the surface layer.

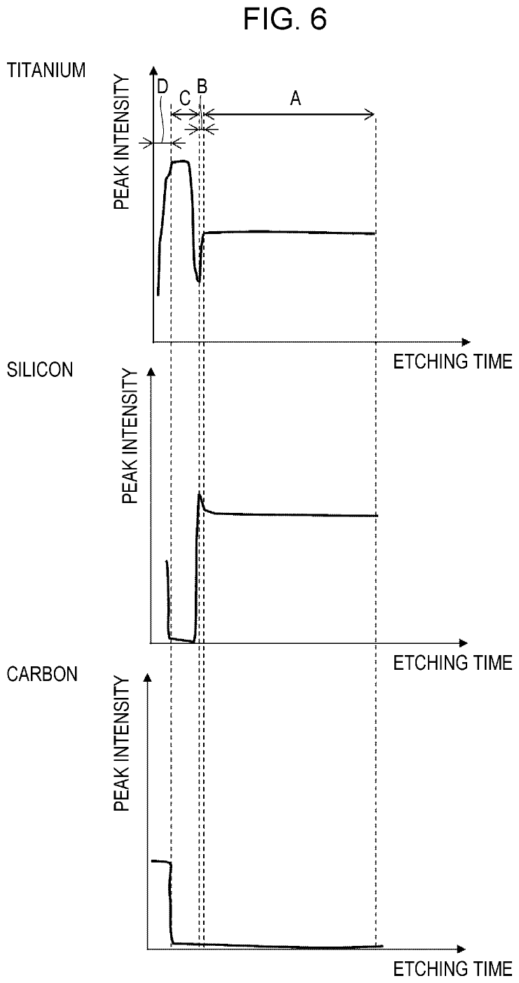

The elemental profiles will be described below with reference to FIG. 6.

FIG. 6 illustrates examples of the elemental profiles of the silica-titania composite aerogel particles and includes, in sequence from top, an elemental profile for titanium, an elemental profile for silicon, and an elemental profile for carbon.

The elemental profiles illustrated in FIG. 6 are divided into a region A, a region B, a region C, a region D at the inflection points of the profile curve.

Region A: a region that is present at the final stage of etching and in which the titanium peak intensity and the silicon peak intensity are substantially constant.

Region B: a region that is present immediately before the region A and in which the closer to the surfaces of the particles, the lower the titanium peak intensity and the higher the silicon peak intensity.

Region C: a region that is present immediately before the region B and in which the titanium peak intensity is substantially constant and silicon is rarely detected.

Region D: a region that is present at the initial stage of etching and in which the carbon peak intensity is substantially constant and a metal element is also detected.

The region A and the region B are regions that reflect the elemental composition of the base particles. In the production of the base particles, the base particles are formed by forming covalent bonds between silica and titania at the ratio corresponding to the mixing ratio of the alkoxy silane and the titanium alkoxide, which are materials of the silica-titania composite. However, silica is more likely to appear on the surfaces of the base particles than titania. As a result, the elemental profiles include the region A, which is present at the final stage of etching and in which the titanium peak intensity and the silicon peak intensity are substantially constant; and the region B, which is present immediately before the region A and in which the closer to the surfaces of the particles, the lower the titanium peak intensity and the higher the silicon peak intensity.

The region C is a region that reflects the elemental composition of the intermediate layer. When the region C, which is a region in which the titanium peak intensity is substantially constant and silicon is rarely detected, is present immediately before the region B, the silica-titania composite aerogel particles are determined to have an intermediate layer, which is a “titania layer”.

The region C reflects the elemental composition of a first layer, but does not necessarily completely correspond to the intermediate layer. A portion of the region C adjacent to the region B may reflect the elemental composition of the base particles.

The region D is a region that reflects the elemental composition of the surface layer. When the region D, which is a region in which the carbon peak intensity is substantially constant and a metal element is also detected, is present at the initial stage of etching, the silica-titania composite aerogel particles are determined to have a surface layer, which is a “layer containing a metal compound having a metal atom and a hydrocarbon group”.

Since silicon, aluminum, and titanium are candidates for the metal atom of the metal compound in the surface layer, the identification and quantitative determination of aluminum are also performed by XPS as needed, and the elemental profile for aluminum is also drawn.

The region D reflects the elemental composition of the surface layer, but does not necessarily completely correspond to a second layer. A portion of the region D adjacent to the region C may reflect the elemental composition of the first layer.

The elemental profiles illustrated in FIG. 6 indicate that the silica-titania composite aerogel particles have base particles, an intermediate layer, and a surface layer, and the metal atom of the metal compound in the surface layer is silicon.

### Characteristics of Filter

In view of filter function, the air permeability of the filter according to the exemplary embodiment is preferably 1 (cm³/cm²·sec) or more and 300 (cm³/cm²·sec) or less, and more preferably 5 (cm³/cm²·sec) or more and 200 (cm³/cm²·sec) or less.

The air permeability of the filter is measured as described below.

The air permeability of the filter is measured with a frazir-type air permeability tester AP-360SM (available from Daiei Kagaku Seiki Mfg. Co., Ltd.). Specifically, a test piece, about 20 cm×20 cm, is attached to an end of the cylinder in the frazir-type air permeability tester, and a suction fan is then controlled with a rheostat so that the inclined manometer shows a pressure of 125 Pa (1.27 cmH₂O). On the basis of the pressure shown by the vertical manometer and the type of vent hole used in this case, the amount (cm³/cm²/s) of air passing through the test piece is obtained from the table attached the tester. The measurement is performed 5 times, and the mean is taken as the air permeability of the filter.

The filter according to the exemplary embodiment may be visible light transmissible in order that the photocatalyst particles having a high photocatalytic function in the visible light region efficiently exhibit a catalytic function.

Specifically, the visible light transmittance of the filter is preferably 30% or more, more preferably 30% or more and 70% or less, and still more preferably 50% or more and 70% or less.

The visible light transmittance of the filter is measured as described below.

The total light transmittance (%) is measured by using a haze meter (NDH-2000 available from Nippon Denshoku Industries Co., Ltd,) in accordance with JIS K7361-1:1997.

The filter according to the exemplary embodiment may have high lightness. This is because, when the filter has high lightness, the reflection of visible light causes the photocatalyst particles having a high photocatalytic function in the visible light region to efficiently exhibit a catalytic function.

Specifically, the L* value of the filter body in the CIE 1976 L*a*b* color space is preferably in the range of 35% or more and 95% or less, more preferably in the range of 45% or more and 90% or less, and still more preferably in the range of 55% or more and 85% or less.

To improve deodorizing performance, the amount of the photocatalyst particles held on the filter body in the filter according to the exemplary embodiment is preferably 3% or more and 30% or less, more preferably 8% or more and 25% or less, and still more preferably 10% or more and 20% or less.

The amount of the photocatalyst particles held on the filter body is determined as described below.

The amount of the photocatalyst particles held on the filter body is determined as follows: measuring the weight of the filter body before and after the photocatalyst particles are held on the filter body; and calculating the amount of the photocatalyst particles in accordance with the following formula.

Amount (%) of photocatalyst particles held on filter body=[(weight of filter body after photocatalyst particles are held on filter body−weight of filter body before photocatalyst particles are held on filter body)/weight of filter body after photocatalyst particles are held on filter body)×100

To prevent or reduce the detachment of the photocatalyst particles from the filter due to an impact or the like, the proportion of the photocatalyst particles released from the filter body in the filter according to the exemplary embodiment is preferably 1% or more and 50% or less, more preferably 3% or more and 40% or less, and still more preferably 5% or more and 30% or less.

When the proportion of the released photocatalyst particles is 1% or more, the deterioration of the photocatalytic effect (i.e., the deterioration of the deodorizing performance of the filter) caused as a result of strong embedding of the photocatalyst particles in the wall surfaces of the pores of the filter body is unlikely to occur.

When the proportion of the released photocatalyst particles is 50% or less, the detachment of the photocatalyst particles from the filter body is prevented or reduced.

The proportion of the released photocatalyst particles is controlled by the particle size of the photocatalyst particles and the conditions under which the photocatalyst particles are held on (attached to) the filter body.

The proportion of the photocatalyst particles released from the filter body is determined as described below.

The filter body (i.e., filter) having the photocatalyst particles is immersed in 40300 ml of a 0.2% aqueous solution of a surfactant (polyoxyethylene octylphenyl ether (available from Wako Pure Chemical Industries, Ltd.). A dispersion is obtained. With the filter body immersed, the photocatalyst particles are then released from the surface of the filter body by applying ultrasonic vibration (output 20 W, frequency 20 kHz) to the dispersion for 1 minute. The supernatant of the dispersion other than the filter body is then removed, and pure water is added, followed by filtering and drying. As a result, the filter body from which the photocatalyst particles have been released is obtained.

The amount of an “atom (e.g., titanium atom) in the photocatalyst particles” of the filter body having the photocatalyst particles before ultrasonic vibration, and the amount of the “atom (e.g., titanium atom) in the photocatalyst particles” of the filter body that are obtained after ultrasonic vibration and from which the photocatalyst particles have been released are measured with fluorescent X-rays, and the proportion of the photocatalyst particles released from the filter body is calculated in accordance with the following formula.

Proportion (%) of photocatalyst particles released from filter body=((amount of “atom (e.g., titanium atom) in photocatalyst particles” of filter body before ultrasonic vibration)−(amount of “atom (e.g., titanium atom) in photocatalyst particles” of filter body after ultrasonic vibration))/(amount of “atom (e.g., titanium atom) in photocatalyst particles” of filter body before ultrasonic vibration)×100

### Method for Producing Filter

Examples of the method for producing the filter according to the exemplary embodiment include, but are not limited to, a method of applying a dispersion of photocatalyst particles to the filter body and then drying the dispersion so that the photocatalyst particles are held on the filter body. Alternatively, the filter may be produced after photocatalyst particles are preliminarily held on a paper material for producing the filter body.

Since the photocatalyst particles formed of the specific titanium compound particles have an adhesive surface, the photocatalyst particles are directly attached and immobilized to the wall surfaces of the pores of the filter body.

The coating method may be a well-known coating method, such as dip coating or spray coating.

Examples of the dispersion medium used for the dispersion include volatile dispersion media, such as water and various alcohols.

Alternatively, a method of causing the filter body to have photocatalyst particles by using a binder resin may be employed.

### EXAMPLES

Exemplary embodiments of the present disclosure will be described below in detail by way of Examples, but exemplary embodiments of the present disclosure are not limited by these Examples. In the following description, the unit “part” is on a mass basis, unless otherwise specified.

### Preparation of Photocatalyst Particles

### Metatitanic Acid Particles MTA1

To a titanyl sulfate solution in which the TiO₂ concentration is 260 g/L and the Ti³⁺ concentration in terms of TiO₂ is 6.0 g/L, a separately prepared anatase seed is added in an amount of 8% by mass in terms of TiO₂ relative to TiO₂ in the titanyl sulfate solution. Next, this solution is heated at the boiling point or higher to hydrolyze titanyl sulfate (TiOSO₄) and thus to generate particle-like metatitanic acid. Next, the metatitanic acid particles are filtered and washed, and a slurry of the metatitanic acid particles is then prepared and subjected to neutralization and washing at pH 7. Accordingly, a metatitanic acid slurry with an average particle size of 0.042 μm is prepared.

Next, a 5 N aqueous solution of sodium hydroxide is added to the metatitanic acid slurry with an average particle size of 0.042 μm under stirring until the pH reaches 8.5. The slurry is maintained under stirring for 2 hours and then neutralized with 6 N hydrochloric acid until the pH reaches 5.8, followed by filtering and washing with water. After washing, water is added again to form a slurry again, and 6 N hydrochloric acid is added to the slurry under stirring until the pH reaches 1.3. The slurry is maintained under stirring for 3 hours. From the slurry, 100 parts by mass of metatitanic acid is separated and continuously heated at 60° C. Under stirring, 30 parts by mass of hexyltrimethoxysilane is added to metatitanic acid. The mixture is stirred for 30 minutes and then neutralized to pH 7 by addition of a 7 N aqueous solution of sodium hydroxide, followed by filtering and washing with water. The residue after filtering and washing with water is spray-dried in a jet dryer at an outlet temperature of 150° C. to produce a dry powder. The obtained dry powder is heated at 280° C. for 90 minutes in an electric furnace with an oxygen concentration (% by volume) of 12% to yield metatitanic acid particles MTA1.

### Metatitanic Acid Particles MTA2

Metatitanic acid particles MTA2 with an average particle size of 0.095 μm are produced in the same manner as for the metatitanic acid particles MTA1 except that the amount of the anatase seed added is 6% by mass.

### Metatitanic Acid Particles MTA3

Metatitanic acid particles MTA3 with an average particle size of 0.150 μm are produced in the same manner as for the metatitanic acid particles MTA1 except that the amount of the anatase seed added is 4% by mass.

### Titanium Oxide Particles TO1

To a dispersion in which commercial anatase titanium oxide particles (“SSP-25 (available from Sakai Chemical Industry Co., Ltd.)”, average particle size 0.010 μm) are dispersed in methanol, 35% by mass of hexyltrimethoxysilane relative to the untreated titanium oxide particles is added dropwise. The mixture is caused to react at 40° C. for 1 hour and then spray-dried at an outlet temperature of 120° C. to produce a dry powder. The obtained dry powder is heated at 290° C. for 1 hour in an electric furnace with an oxygen concentration (% by volume) of 18% to yield titanium oxide particles TO1.

### Titanium Oxide Particles TO2

To a dispersion in which commercial anatase titanium oxide particles (“ST-21 (available from Ishihara Sangyo Kaisha, Ltd.)”, average particle size 0.020 μm) are dispersed in methanol, 30% by mass of octyltrimethoxysilane relative to the untreated titanium oxide particles is added dropwise. The mixture is caused to react at 40° C. for 1 hour and then spray-dried at an outlet temperature of 120° C. to produce a dry powder. The obtained dry powder is heated at 270° C. for 1 hour in an electric furnace with an oxygen concentration (% by volume) of 20% to yield titanium oxide particles TO2.

### Titanium Oxide Particles TO3

To a dispersion that is prepared by a sol-gel method and in which anatase titanium oxide particles with an average particle size of 0.450 μm are dispersed in methanol, 25% by mass of hexyltrimethoxysilane relative to the untreated titanium oxide particles is added dropwise. The mixture is caused to react at 40° C. for 1 hour and then spray-dried at an outlet temperature of 120° C. to produce a dry powder. The obtained dry powder is heated at 300° C. for 1 hour in an electric furnace with an oxygen concentration (% by volume) of 18% to yield titanium oxide particles TO3.

### Titanium Oxide Aerogel Particles TOAG1

To a reaction container, 115.4 parts of methanol and 14.3 parts of tetrabutoxy titanium are placed and mixed. While the mixture is stirred at 100 rpm with a magnetic stirrer, 7.5 parts of a 0.009% by mass aqueous solution of oxalic acid is added dropwise to the mixture over 30 seconds. The mixture is maintained under stirring for 30 minutes to form 137.3 parts of a dispersion (1) (solid content: 3.4 parts, liquid phase: 133.9 parts).

Next, 137.3 parts of the dispersion (1) is placed in a reaction vessel, and the temperature and pressure are increased to 150° C./20 MPa by introducing CO₂ to the reaction vessel with the dispersion (1) being stirred at 85 rpm. While the dispersion (1) is continuously stirred, CO2 is caused to flow in and flow out, and 133 parts of the liquid phase is removed over 60 minutes.

Next, to the solid phase that remains after the liquid phase is removed, a mixture of 3.4 parts of isobutyltrimethoxysilane and 3.4 parts of methanol is added over 5 minutes. The resultant mixture is maintained at 150° C./20 MPa for 30 minutes under stirring at 85 rpm. While the mixture is continuously stirred, CO₂ is caused to flow in and flow out, and 6.5 parts of the liquid phase is removed over 30 minutes. The pressure is reduced to the atmospheric pressure over 30 minutes, and 4.6 parts of a powder is collected.

Next, 4.0 parts of the powder is measured out in a SUS container and heated at 315° C. for 60 minutes in an electric furnace with an oxygen concentration (% by volume) of 20%. The powder is allowed to cool to 30° C. The obtained powder is filtered through a vibrating screen with a mesh size of 45 μm to remove coarse particles and, as a result, 3.5 parts of a powder (titanium oxide aerogel particles TOAG1) is collected.

### Silica-Titania Composite Aerogel Particles STAG1

To a reaction container, 115.4 parts of methanol and 7.2 parts of tetramethoxysilane are placed and mixed. To the reaction container, 7.2 parts of tetrabutoxy titanium is further placed and mixed. While the mixture is stirred with a magnetic stirrer at 100 rpm, 7.5 parts of a 0.009% by mass aqueous solution of oxalic acid is added dropwise to the mixture over 30 seconds. The mixture is maintained under stirring for 30 minutes to form 137.2 parts of a first dispersion (I-1) (solid content: 4.5 parts, liquid phase: 132.7 parts).

Next, 137.2 parts of the first dispersion (I-1) is placed in a reaction container, and the temperature and pressure are increased to 150° C./20 MPa by introducing CO₂ to the reaction container with the first dispersion (I-1) being stirred at 85 rpm. While the first dispersion (I-1) is continuously stirred, CO₂ is caused to flow in and flow out, and 132.0 parts of the liquid phase is removed over 60 minutes.

Next, to the solid phase that remains after the liquid phase is removed, a mixture of 4.5 parts of isobutyltrimethoxysilane and 4.5 parts of methanol is added over 5 minutes. The resultant mixture is maintained at 150° C./20 MPa for 30 minutes under stirring at 85 rpm. While the mixture is continuously stirred, CO₂ is caused to flow in and flow out, and 8.2 parts of the liquid phase is removed over 30 minutes. The pressure is reduced to the atmospheric pressure over 30 minutes, and 6.0 parts of a powder is collected.

Next, 4.0 parts of the powder is measured out in a SUS container and placed on a hot plate. The powder is heated to 380° C., maintained for 60 minutes, and allowed to cool to 30° C. The obtained powder is filtered through a vibrating screen with a mesh size of 45 μm to remove coarse particles and, as a result, 3.5 parts of a powder (silica-titania composite aerogel particles STAG1) is collected.

The silica-titania composite aerogel particles STAG1 contain base particles having an elemental ratio Si/Ti of silicon to titanium of 3.1, and an isobutylsilyl group containing surface layer present on the surfaces of the base particles.

### Silica-Titania Composite Aerogel Particles STAG2

To a reaction container, 115.4 parts of methanol and 7.2 parts of tetramethoxysilane are placed and mixed. To the reaction container, 7.2 parts of tetrabutoxy titanium is further placed and mixed. While the mixture is stirred at 100 rpm with a magnetic stirrer, 7.5 parts of a 0.009% by mass aqueous solution of oxalic acid is added dropwise to the mixture over 30 seconds. The mixture is maintained under stirring for 30 minutes to form 137.2 parts of a first dispersion (I-1) (solid content: 4.5 parts, liquid phase: 132.7 parts).

Next, 137.2 parts of the first dispersion (I-1) is placed in a reaction container. While the first dispersion (I-1) is stirred at 100 rpm with a magnetic stirrer, a mixture of 1.5 parts of tetrabutoxy titanium and 4.5 parts of butanol is added dropwise over 10 minutes. The mixture is maintained under stirring for 30 minutes to form 143.2 parts of a second dispersion (II-1) (solid content: 5.0 parts, liquid phase: 138.2 parts).

Next, 143.2 parts of the second dispersion (II-1) is placed in a reaction vessel, and the temperature and pressure are increased to 150° C./20 MPa by introducing CO₂ to the reaction vessel with the second dispersion (II-1) being stirred at 85 rpm. While the second dispersion (II-1) is continuously stirred, CO₂ is caused to flow in and flow out, and 138 parts of the liquid phase is removed over 60 minutes.

Next, to the solid phase that remains after the liquid phase is removed, a mixture of 4.5 parts of isobutyltrimethoxysilane and 4.5 parts of methanol is added over 5 minutes. The resultant mixture is maintained at 150° C./20 MPa for 30 minutes under stirring at 85 rpm. While the mixture is continuously stirred, CO₂ is caused to flow in and flow out, and 7.0 parts of the liquid phase is removed over 30 minutes. The pressure is reduced to the atmospheric pressure over 30 minutes, and 7.2 parts of a powder is collected.

Next, 4.0 parts of the powder is measured out in a SUS container and placed on a hot plate. The powder is heated to 450° C., maintained for 60 minutes, and allowed to cool to 30° C. The obtained powder is filtered through a vibrating screen with a mesh size of 45 μm to remove coarse particles and, as a result, 3.5 parts of a powder (silica-titania composite aerogel particles STAG2) is collected.

The silica-titania composite aerogel particles STAG2 contain base particles having an elemental ratio Si/Ti of silicon to titanium of 3.1, a titania layer (intermediate layer) present on the surfaces of the base particles, and an isobutylsilyl group containing surface layer present on the surface of the titania layer.

The following characteristics of the photocatalyst particles produced as described above are determined in accordance with the above-described methods. The photocatalyst particles are listed in Table 1.
Visible Absorption Spectrum Characteristics (expressed as “Visi Characteristics” in Table: the absorbance at a wavelength of 450 nm, the absorbance at a wavelength of 600 nm, and the absorbance at a wavelength of 750 nm, assuming that the absorbance at a wavelength of 350 nm is 1)
Infrared Absorption Spectrum Characteristics (expressed as “IR Characteristics” in Table: the presence or absence of absorption peaks in the wavenumber range of 2700 cm⁻¹ or more and 3,000 cm or less, and the wavenumber of the absorption peaks)
Average Particle Size (expressed as “Particle Average Size DC” in Table)

### Production of Filters F1 to F4

The following filter bodies (size: 55 mm long×55 mm wide×10 mm thick) are provided. The details of the provided filter bodies are described in Table 2.
Filter body F1 (trade name “HONEYCOMB-P KLA-100 (available from Shin Nippon Feather Core Co., Ltd.)”, honeycomb paper filter)
Filter body F2 (trade name FEATHER CORE F-R available from Shin Nippon Feather Core Co., Ltd., honeycomb paper filter)
Filter body F3 (trade name “NB CORE (available from Nagoya Core Co., Ltd.)”, honeycomb paper filter)
Filter body F4 (paper filter obtained by forming Sinar semi-bleached kraft (available from APP Japan Limited.) into a honeycomb shape)

A dispersion of photocatalyst particles in ethanol (the total number of parts of the dispersion is assumed to 100 parts) is prepared with the type and the number of parts shown in Table 3.

The obtained dispersion is sprayed onto the filter body shown in Table 3 and dried and, as a result, the photocatalyst particles are held on the filter body. The amount of the dispersion sprayed is controlled in such a manner that the amount of the held photocatalyst particles is the amount shown in Table 3.

The filters FT1 to FT12 are produced accordingly.

### Production of Comparative Filters

### Comparative Filters CFT1 and CFT2

Comparative filters CFT1 and CFT2 are produced in the same manner as for the filter FT1 except that the following comparative filter bodies (size: 55 mm long×55 mm wide×10 mm thick) are used. The details of the provided filter bodies are described in Table 2.
Comparative filter body CF1: a paper filter obtained by making paper using hardwood pulp with an average fiber diameter of 5 μm and forming the paper so as to have a honeycomb structure.
Comparative filter body CF2: a paper filter obtained by making paper using softwood pulp with an average fiber diameter of 70 μm and forming the paper so as to have a honeycomb structure.

### Comparative Filter CFT3

A comparative filter CFT3 is produced in the same manner as for the filter FT1 except that commercial titanium oxide particles (trade name “ST-01 (available from Ishihara Sangyo Kaisha, Ltd.), catalyst particles having an average particle size of 0.012 μm and having no visible light-driven photocatalytic function) are used as photocatalyst particles.

### Evaluation

### Evaluation of Characteristics

The following characteristics of the obtained filters are measured in accordance with the above-described methods.
Air permeability (cm³/cm²·sec) of filter
Visible light transmittance (%) of filter
L* value of filter body in CIE 1976 L*a*b* color space
Proportion (%) of photocatalyst particles released from filter body

### Evaluation of Deodorization Maintainability

A visible light-type photocatalyst air cleaning device is produced by replacing LEDs for ultraviolet radiation in a commercial compact air cleaner (trade name “LED PURE AH1 available from Nitride Semiconductor Co., Ltd.)” with LEDs for visible-light radiation (constant current driver-embedded triple white LED module AE-LED1×3-12V (available from Akizuki Denshi Tsusho Co., Ltd.)).

The filters FT1 to FT12 and CFT1 to CFT3 in examples are set at a deodorizing filter installation position in the visible light-type photocatalyst air cleaning device.

The decomposition of ammonia is checked by using this device in the following manner.

The visible light-type photocatalyst air cleaning device is placed in an acrylic vacuum desiccator with a volume of 7 L, and the acrylic vacuum desiccator is sealed. Subsequently, ammonia gas is introduced into the desiccator until the gas concentration reaches 280 ppm, and the air cleaning device is started. The air in the desiccator is sampled at regular time intervals. The concentration of ammonia gas is measured by using an ammonia gas detector tube, and the deodorization decomposition performance is evaluated.

The decomposition of ammonia is checked after a device operation time of 0.2 hours (early stage) and after a device operation time of 3.5 hours (elapsed time) and evaluated on the basis of the following criteria.
A: The ammonia concentration is less than 1.0 ppm.
B: The ammonia concentration is 1.0 ppm or more and less than 5.0 ppm.
C: The ammonia concentration is 5.0 ppm or more and less than 10 ppm.
D: The ammonia concentration is 10 ppm or more and less than 50 ppm.
E: The ammonia concentration is 50 ppm or more.

### Evaluation of Detachment of Photocatalyst Particles

The proportion of the photocatalyst particles detached from the filter is evaluated in accordance with the following method.

The Ti element content of the filter before evaluation of deodorization maintainability and after a device operation time of 3.5 hours (elapsed time) in the evaluation of deodorization maintainability is measured with fluorescent X-rays, and the proportion of detached photocatalyst particles is evaluated.

Proportion (%) of detached photocatalyst particles=(Ti element content before evaluation of deodorizing performance—Ti element content after evaluation of deodorizing performance)/Ti element content before evaluation of deodorizing performance×100

The evaluation criteria are as described below.
A: The proportion of detached photocatalyst particles is less than 5%.
B: The proportion of detached photocatalyst particles is 5% or more and less than 10%.
C: The proportion of detached photocatalyst particles is 10% or more and less than 20%.
D: The proportion of detached photocatalyst particles is 20% or more and less than 30%.
E: The proportion of detached photocatalyst particles is 30% or more.

The details and the evaluation results of the filters are summarized in Table 3.

In the overall evaluation in Table 3, examples that receive at least one grade A among all evaluation items are rated A (⊙), examples that receive grade B or C are rated B (O), and examples that receive grade D or E are rated C (x).

TABLE 1

| Photocatalyst Particles | | | | | |
|---|---|---|---|---|---|
| | Particle | Visi Characteristics | | | |
| | Average | Absorbance | Absorbance | Absorbance | IR Characteristics |
| | Size DC | at Wavelength | at Wavelength | at Wavelength | Wavenumber (cm⁻¹) |
| | (μm) | of 450 nm | of 600 nm | of 750 nm | of Absorption Peak |
| Metatitanic Acid Particles MTA1 | 0.042 | 0.63 | 0.46 | 0.29 | 2850/2920 |
| Metatitanic Acid Particles MTA2 | 0.095 | 0.58 | 0.42 | 0.23 | 2854/2924 |
| Metatitanic Acid Particles MTA3 | 0.150 | 0.44 | 0.30 | 0.19 | 2848/2918 |
| Titanium Oxide Particles TO1 | 0.010 | 0.62 | 0.37 | 0.28 | 2849/2917 |
| Titanium Oxide Particles TO2 | 0.020 | 0.55 | 0.33 | 0.21 | 2853/2917 |
| Titanium Oxide Particles TO3 | 0.450 | 0.35 | 0.21 | 0.10 | 2852/2919 |
| Titanium Oxide Aerogel Particles | 0.080 | 0.60 | 0.37 | 0.25 | 2855/2920 |
| TOAG1 | | | | | |
| Silica-Titania Composite Aerogel | 0.024 | 0.24 | 0.15 | 0.06 | 2847/2921 |
| Particles STAG1 | | | | | |
| Silica-Titania Composite Aerogel | 0.056 | 0.25 | 0.16 | 0.06 | 2851/2923 |
| Particles STAG2 | | | | | |

TABLE 2

| | | Average | Porosity | | Average | Average |
|---|---|---|---|---|---|---|
| | Surface | Opening | (%) | | Fiber | Fiber |
| | Roughness | Diameter | of | Basis | Length | Diameter |
| Filter | Ra | (mm) of | Opening | Weight | (mm) of | (μm) of |
| Body | (μm) | Openings | Plane | (g/m²) | Pulp Fiber | Pulp Fiber |
| F1 | 3.3 | 12 | 55 | 70 | 3.5 | 30 |
| F2 | 4.8 | 10.5 | 70 | 170 | 3.8 | 25 |
| F3 | 3.5 | 5 | 38 | 100 | 3.8 | 33 |
| F4 | 3.7 | 2.5 | 40 | 70 | 3.5 | 30 |
| CF1 | 0.8 | 20 | 85 | 70 | 3.5 | 30 |
| CF2 | 20 | 0.5 | 15 | 70 | 3.5 | 30 |

TABLE 3

| | Photocatalyst | | | | | | | | |
|---|---|---|---|---|---|---|---|---|---|
| | Particles | | | | Characteristics | | | | |
| | | Particle | Filter Body | | Amount (%) | | | | |
| | | Average | | Surface | of Held | | | Air | Visible Light |
| | | Size DC | | Roughness | Photocatalyst | DC/Surface | DC/Fiber | Permeability | Transmittance |
| | Type | (μm) | Type | (μm) | Particles | Roughness | Diameter | (cm³/cm² · sec) | (%) |
| FT1 | MTA1 | 0.042 | F1 | 3.3 | 20 | 0.013 | 0.0004 | 120 | 55 |
| FT2 | MTA2 | 0.095 | F1 | 3.3 | 20 | 0.029 | 0.0010 | 145 | 58 |
| FT3 | MTA3 | 0.150 | F1 | 3.3 | 20 | 0.045 | 0.0015 | 180 | 59 |
| FT4 | TO1 | 0.010 | F1 | 3.3 | 20 | 0.003 | 0.0001 | 55 | 53 |
| FT5 | TO2 | 0.020 | F1 | 3.3 | 20 | 0.006 | 0.0002 | 65 | 53 |
| FT6 | TO3 | 0.450 | F1 | 3.3 | 20 | 0.136 | 0.0055 | 70 | 60 |
| FT7 | TOAG1 | 0.080 | F1 | 3.3 | 20 | 0.024 | 0.0008 | 98 | 54 |
| FT8 | STAG1 | 0.024 | F1 | 3.3 | 20 | 0.007 | 0.0002 | 48 | 53 |
| FT9 | STAG2 | 0.056 | F1 | 3.3 | 20 | 0.017 | 0.0006 | 58 | 56 |
| FT10 | MTA1 | 0.042 | F2 | 4.8 | 20 | 0.009 | 0.0004 | 105 | 55 |
| FT11 | MTA1 | 0.042 | F3 | 3.5 | 20 | 0.012 | 0.0004 | 112 | 54 |
| FT12 | MTA1 | 0.042 | F4 | 3.7 | 20 | 0.011 | 0.0004 | 110 | 55 |
| CFT1 | MTA1 | 0.042 | CF1 | 0.8 | 20 | 0.047 | 0.0004 | 270 | 25 |
| CFT2 | MTA1 | 0.042 | CF2 | 20 | 20 | 0.004 | 0.0004 | 15 | 85 |
| CFT3 | ST-01 | 0.012 | F1 | 3.3 | 20 | 0.004 | 0.0004 | 95 | 50 |

| | Characteristics | | | | | | |
|---|---|---|---|---|---|---|---|
| | | Proportion | Evaluation | | | | |
| | | (%) | Deodorization | | Detachment | | |
| | L* | of Released | Maintainability | | of | | |
| | Value | Photocatalyst | Early | Elapsed | Photocatalyst | Overall | |
| | (%) | Particles | Stage | Time | Particles | Evaluation | Note |
| FT1 | 72 | 14 | B | C | A | A (⊚) | Example |
| FT2 | 74 | 17 | C | B | A | A (⊚) | Example |
| FT3 | 76 | 30 | C | B | B | B (◯) | Example |
| FT4 | 71 | 23 | C | C | A | A (⊚) | Example |
| FT5 | 74 | 29 | C | B | A | A (⊚) | Example |
| FT6 | 75 | 33 | C | A | B | A (⊚) | Example |
| FT7 | 72 | 19 | B | A | A | A (⊚) | Example |
| FT8 | 80 | 24 | B | B | A | A (⊚) | Example |
| FT9 | 80 | 32 | B | B | B | B (◯) | Example |
| FT10 | 72 | 13 | C | B | A | A (⊚) | Example |
| FT11 | 72 | 13 | C | B | B | B (◯) | Example |
| FT12 | 72 | 14 | B | A | B | A (⊚) | Example |
| CFT1 | 70 | 30 | D | D | B | C (X) | Comparative |
| | | | | | | | Example |
| CFT2 | 69 | 34 | E | D | C | C (X) | Comparative |
| | | | | | | | Example |
| CFT3 | 90 | 35 | C | B | D | C (X) | Comparative |
| | | | | | | | Example |

The above-described results indicate that the filters according to Examples have higher deodorization maintainability than the filters according to Comparative Examples.

The above-described results also indicate that the filters according to Examples have higher deodorizing performance than the filters according to Comparative Examples.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments was/were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. A filter comprising:
a filter body containing a paper sheet having a surface roughness of 1.0 μm or more and 10.0 μm or less; and
each photocatalyst particle of photocatalyst particles, held on the filter body and having a surface to which a metal compound having a metal atom and a hydrocarbon group is bonded via an oxygen atom, the photocatalyst particle being formed of a titanium compound particle having absorption at wavelengths of 450 nm and 750 nm in a visible absorption spectrum.

2. The filter according to claim 1, wherein the photocatalyst particle has absorption in an entire wavelength range from 400 nm to 800 nm in the visible absorption spectrum.

3. The filter according to claim 1, wherein the photocatalyst particle has an absorption peak in a range from 2700 cm⁻¹ to 3000 cm⁻¹ in an infrared absorption spectrum.

4. The filter according to claim 1, wherein the photocatalyst particle is at least one type of particle selected from the group consisting of a metatitanic acid particle, a titanium oxide particle, a titanium oxide aerogel particle, and a silica-titania composite aerogel particle.

5. The filter according to claim 1, wherein the photocatalyst particles have an average particle size in a range from 0.01 μm to 0.5 μm.

6. The filter according to claim 1, wherein the photocatalyst particles have an average particle size in a range from 0.02 μm to 0.15 μm.

7. The filter according to claim 1, wherein a ratio (average particle size/surface roughness) of an average particle size of the photocatalyst particles to a surface roughness of the filter body is in a range from 0.001 to 0.5.

8. The filter according to claim 1, wherein a ratio (average particle size/fiber diameter) of an average particle size of the photocatalyst particles to a fiber diameter of the paper sheet of the filter body is in a range from 0.0001 to 0.1.

9. The filter according to claim 1, wherein the photocatalyst particle is a metatitanic acid particle.

10. The filter according to claim 1, wherein the filter has a visible light transmittance in a range from 30% to 70%.

11. The filter according to claim 1, wherein the filter body has a L* value in a range from 35% to 95% in a CIE 1976 L*a*b* color space.
